(19) United States
(12) Patent Application Publication
(10) Pub. No.: US 2025/0129116 A1
(43) Pub. Date: Apr. 24, 2025

(54) CYCLOPROPANOL AS A BIOORTHOGONAL WARHEAD FOR ELECTROCHEMICALLY CONTROLLED BIOCONJUGATION APPLICATIONS

(71) Applicant: UNIVERSITY OF SOUTH CAROLINA, COLUMBIA, SC (US)

(72) Inventors: Qian Wang, COLUMBIA, SC (US); SHAN HU, COLUMBIA, SC (US)

(21) Appl. No.: 18/921,652

(22) Filed: Oct. 21, 2024

Related U.S. Application Data
(60) Provisional application No. 63/591,514, filed on Oct. 19, 2023.

(51) Int. Cl. C07K 1/13; C25B 3/23; G01N 33/58

(52) U.S. Cl. CPC C07K 1/13; C25B 3/23; G01N 33/582

(57) ABSTRACT

In general, disclosed herein are methods for producing a chemoselectively modified biomolecule. The method may include providing a biomolecule, which may include a bioconjugation warhead at a specific site of the biomolecule. Also, the method may include selectively oxidizing the halogen donor compound of the bioconjugation warhead at an electrochemically efficient voltage to generate a halogen radical. Also, the method may include contacting the halogen radical with the bioconjugation warhead at a constant voltage to form an electrophilic warhead. Also, the method may include reacting the electrophilic warhead with a coupling partner comprising a nucleophilic group of the biomolecule to conjugate the electrophilic warhead to the biomolecule.

### CROSS REFERENCE TO RELATED APPLICATIONS

This application claims the filing benefit of U.S. Provisional Patent Application Ser. No. 63/591,514, filed on Oct. 19, 2023, which is incorporated herein by reference.

### GOVERNMENT SUPPORT CLAUSE

This invention was made with government support under Grant No. 2216137 and Grant No. 2051699 awarded by the National Science Foundation. The government has certain rights in the invention.

### BACKGROUND

Bioconjugation techniques play an important role in modern chemical biology and biomedical sciences, such as protein drug stabilization, target therapy, and the development of antibody-drug conjugates, etc. Typically, bioorthogonality is required for an effective in situ bioconjugation reaction, which enables the selective and non-disruptive labeling and manipulation of biomolecules within complex living systems. On the other hand, the size of the linker group, also termed as a bioconjugation warhead, should be small enough to minimize the perturbation of biological processes. Therefore, azide, alkyne, diazirine, cyclopropene, and oxaziridine derivatives, have been widely used in bioconjugation reactions due to their bioorthogonal reactivities and small functional group size. While the reigning of click chemistry has boosted the application of azides and alkynes, making them the most popular molecules for bioconjugation applications, three-member ring systems have emerged as another type of powerful warhead in recent years.

Diazirine was the first reported photoactive warhead with a three-member ring system developed for bioconjugation. Upon exposure to light irradiation, it can form highly reactive carbenes capable of engaging various functional groups either in vitro or in vivo, making it widely used to study lipid-protein interactions via photoaffinity labeling.

Previously, an approach was developed for in vivo photoaffinity labeling that uses unnatural diazirine labeling cholesterols or choline and in situ generated photoactivable glycerophospholipids. After that, the synthesis and biosynthetic incorporation of unnatural photoactivatable amino acids or monosaccharides containing diazirine crosslinkers into live cells has been extensively explored and applied to investigate glycoprotein or protein-protein interactions within live cells.

Cyclopropene is another bioconjugation warhead with a three-member ring system commonly used in bioconjugation applications. The development of this minimal chemical reporter was inspired by the limitations posed by the presence of steric labeling tags such as trans-cyclooctene and bicyclononyne in biological environments. Thus, cyclopropene, as a substitution for steric and strained labeling tags, can also react robustly with tetrazine probes via cycloaddition reactions under physiological environments. For example, the methylcyclopropene-sialic acid conjugate was synthesized, which was successfully incorporated into cell surface glycans to facilitate visualization of targeted biomolecular interactions in vivo through specific reactions with a tetrazine probe.

Furthermore, a redox-activated chemical tagging (ReACT) was developed with a member ring system, oxaziridine, that enables chemoselective methionine bioconjugation in proteins and proteomes via oxidative sulfur imidation reaction. The oxaziridine warhead has been widely applied in the applications of antibody-drug conjugates (ADCs), using the antibody fragment to a green fluorescent protein (GFP-Fab) as a starting model, and activity-based protein profiling (ABPP) of oncoprotein cyclin-dependent kinase 4 (CDK4).

Despite the remarkable achievements of these three-member ring systems in bioconjugation, the options for utilizing the cyclopropene warhead in bioconjugation remain limited, the diazirine tag exhibits reduced chemical specificity due to its heightened reactivity upon exposure to light, and the size of oxaziridine substitution increased the potential for interference with live cells. Collectively, these limitations underscore the need to develop an improved bioconjugation reporter to enhance its applicability and selectivity in biological applications.

### SUMMARY

In general, disclosed herein are methods for producing a chemoselectively modified biomolecule. The method may include providing a biomolecule, which may include a bioconjugation warhead at a specific site of the biomolecule; selectively oxidizing a halogen donor compound of the bioconjugation warhead at an electrochemically efficient voltage to generate a halogen radical; contacting the halogen radical with the bioconjugation warhead at a constant voltage to form an electrophilic warhead; and reacting the electrophilic warhead with a coupling partner comprising a nucleophilic group of the biomolecule to conjugate the electrophilic warhead to the biomolecule.

Also, disclosed herein is a conjugated biomolecule. The conjugated biomolecule may include an electrophilic warhead conjugated to a specific site of a biomolecule. The electrophilic warhead may include the following formula:

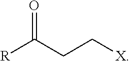

Also, disclosed herein are methods for imaging cells. The method of imaging cells may include incubating the cells with a bioconjugation warhead for a sufficient time to allow for metabolic incorporation of the bioconjugation warhead;. electrochemically promoting the formation of conjugated biomolecules within live cells; labeling conjugated biomolecules with a dye; and imaging cells comprising the conjugated biomolecule and the dye.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates a reaction scheme of cyclopropanol (CPol) derivatives in a divided H-cell apparatus, and proposed ring opening mechanism. RE is a reference electrode.

FIG. 2A depicts screening of CPol derivatives (0.2 mmol) with NaI (0.36 mmol, 1.8 eq.) and NaBr (0.36 mmol, 1.8 eq.) individually under constant voltage.

FIG. 2B depicts Cyclic Voltammetry (CV) curves of 0.5 mmol anions in mixed solvents (ACN/H₂O=1:1, containing 0.1 M Na₂SO₄).

FIG. 2C depicts Cyclic Voltammetry curves of various CPol derivatives (0.2 mmol) in mixed solvents (ACN/H₂O=1:1, containing 0.1 M Na₂SO₄).

FIG. 3A depicts a reaction scheme between phenylcyclopropanol I-1 and different bromo-donors under the same electrochemical conditions.

FIG. 3B depicts Cyclic Voltammetry curves of 0.36 mmol anions in mixed solvents (ACN/H₂O=1:1, containing 0.1 M Na₂SO₄).

FIG. 4A depicts bioconjugation scheme under electrochemical conditions (0.1 M phosphate-buffered saline, pH 7.0).

FIG. 4B depicts MALDI-TOF MS analysis of conjugation of Bivalirudin (M=2179 Da with I-1 and NaBr under a constant voltage of 1.2 V, and LC-MS analysis of the modified amino acid site on Bivalirudin).

FIG. 4C depicts MALDI-TOF MS analysis of conjugation of Angiotensin I (M=1296 Da) with I-1 and NaBr under a constant voltage of 1.2 V, and LC-MS analysis of the modified amino acid site on Angiotensin I.

FIG. 4D depicts MALDI-TOF MS analysis of conjugation of Lysozyme (M=14.3 kDa) with various CPol derivatives (I-1, I-4, and I-7) and NaBr under a constant voltage of 1.2 V and potassium phosphate buffer (0.1 M, pH 7.0).

FIG. 4E depicts MALDI-TOF MS analysis of conjugation of Bovine Serum Albumin (BSA) (M=65.7 kDa) with various CPol derivatives (1 and 4) and NaX (X═I or Br) under a constant voltage of 0.8 V or 1.2 V and potassium phosphate buffer (0.1 M, pH 7.0).

FIG. 4F depicts MALDI-TOF MS analysis of conjugation of wild-type tobacco mosaic virus (wt-TMV) (M=17.5kDa) with I-4 and NaBr under a constant voltage of 1.2 V, and the conjugation of the conjugated wt-TMV with biotin-oxyamine under phosphate buffer (0.1 M, pH 5.8). FIG. 5A depicts the synthetic pathway of natural or unnatural phosphatidylcholine (PC) and Sphingomyelin (SM) following the metabolic incorporation of CPol-choline analog into mammalian cells.

FIG. 5B depicts the incorporation efficiency of propylcyclopropanol choline (12, PCPol-Cho), which was calculated using the ratio of extracted ion chromatograms (XICs) of the native PC headgroup to that of PCPol-PC headgroup.

FIG. 6A depicts scheme of metabolic incorporation of CPol-choline analog into Vero cells and analysis of cellular labeling.

FIG. 6B depicts measurement of intracellular fluorescence intensity after labeling with dye fluorescein thiosemicarbazide (FTSCA) by plate reader.

FIG. 6C depicts average corrected total cell fluorescence (CTCF) intensity was measured from Vero cell images measured by fluorescence microscopy.

FIG. 6D depicts imaging choline-containing phospholipids in Vero cells with CPol choline. Vero cells, labeled with Cpol (5 mM) and treated with NaI (100 μM) under constant voltage (0.7 V), were fixed with 70% ethanol solution and then stained with 50 μM FTSCA.

FIG. 7 depicts optimization screening of CPol derivatives (0.5 mmol) with various amounts of NaBr under constant voltage.

FIG. 8 depicts NMR spectra of Formula I crude product.

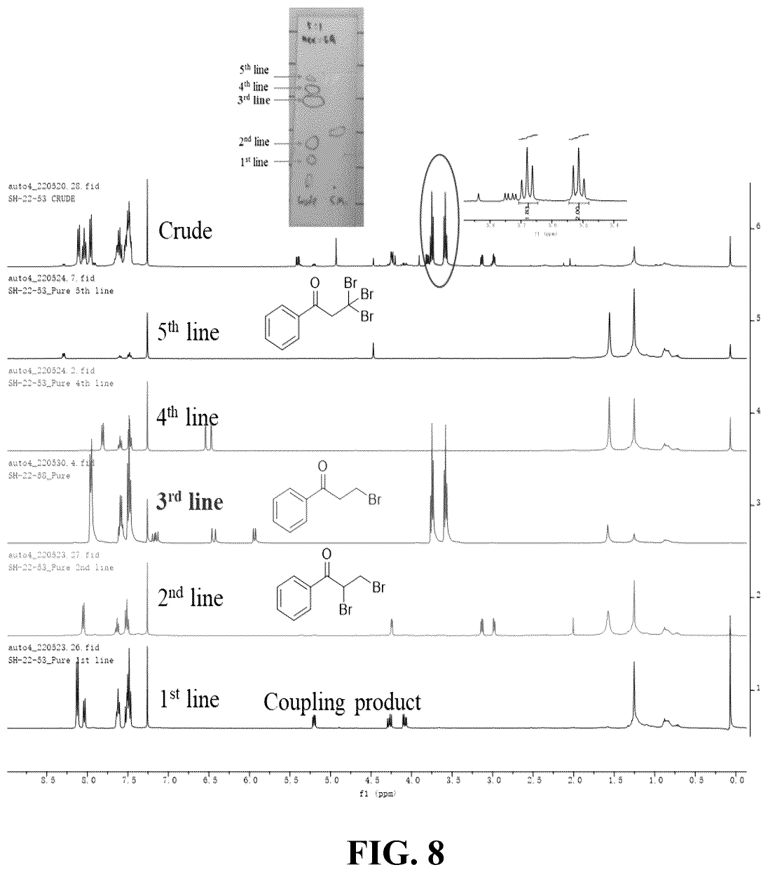

FIG. 9 depicts MALDI-TOF MS spectra of TMV conjugation under various conditions.

FIG. 10 depicts MALDI-TOF MS spectra of bivalirudin conjugation under the electrochemical condition.

FIG. 11 depicts MALDI-TOF MS spectra of bivalirudin conjugation without electrochemical conditions.

FIG. 12 depicts MALDI-TOF MS spectra of lysozyme conjugation without electrochemical conditions.

FIG. 13 depicts cytotoxicity of choline analogs on Vero cells.

FIG. 14 depicts cytotoxicity of NaI and iodine on Vero cells.

FIG. 15A depicts lipidomic experiments performed on lipids extracted from CPol-Cho (5 mM) administrated mammalian cells.

FIG. 15B depicts the incorporation yield calculation approach.

FIG. 16 depicts mass spectrometry analysis of fluorescein thiosemicarbazide (FTSCA).

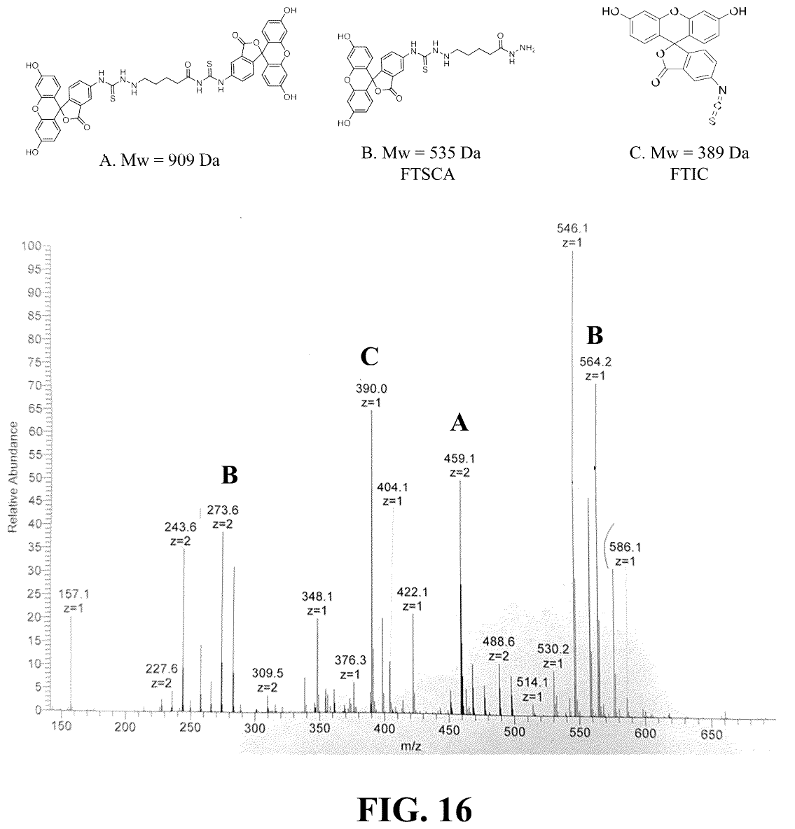

Repeat use of reference characters in the present specification and drawings is intended to represent the same or analogous features or elements of the present invention.

### DETAILED DESCRIPTION

### Definitions

It is to be understood that the terminology used herein is for the purpose of describing particular embodiments only and is not intended to limit the scope of the present disclosure. In this specification and in the claims that follow, reference will be made to a number of terms that shall be defined to have the following meanings.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

As used herein, an “alkyl” group may refer to a straight or branched chain hydrocarbon, having a certain number of carbon atoms (e.g., C₁₋₁₂ carbon atoms). For instance, an alkyl group may include, but is not limited to, straight and branched chain alkyl groups having 1, 2, 3, 4, 5, 6, 7, 8, 9, 10, 11 or 12 carbon atoms. Alternatively, an alkyl group may include, but is not limited to, methyl, ethyl, n-propyl, isopropyl, n-butyl, t-butyl, isobutyl, sec-butyl, n-pentyl, n-hexyl, n-heptyl, n-octyl, or a combination thereof.

As used herein, an “aryl” group may refer to, by itself or as part of another substituent, may include, but is not limited to, a monocyclic, bicyclic or polycyclic polyunsaturated aromatic hydrocarbon radical containing 6 to 14 ring carbon atoms, which may be a single ring or multiple rings (up to three rings) which are fused together or linked covalently.

As used herein, an “alkenyl” group may refer to a linear monovalent hydrocarbon radical or a branched monovalent hydrocarbon radical having the number of carbon atoms indicated in the prefix and containing at least one double bond. Additionally, as used herein, “alkynyl” may refer to a linear monovalent hydrocarbon radical or a branched monovalent hydrocarbon radical containing at least one triple bond and having the number of carbon atoms indicated in the prefix.

As used herein, an “alkoxy” group may refer to an —O-alkyl group, where alkyl is as defined herein. Similarly, an “halogen substituted alkoxy” may refer to an alkoxy in which the alkyl group is substituted with one or more halogen atoms. Additionally, “thioalkoxy” group may refer to an —O-alkylthio group, where the alkylthio group may include, but is not limited to, thiomethoxy, thioethoxy, and the like.

As used herein, a “halogen” group may refer to an element found in Group 17 (formerly Group VIIA) of the periodic table. For instance, a halogen may include, but is not limited to, fluorine (F), chlorine (Cl), bromine (Br), iodine (I), astatine (At), and the like.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

In general, disclosed herein are methods for producing a chemoselectively modified biomolecule. The method may include providing a biomolecule, which may include a bioconjugation warhead at a specific site of the biomolecule. Also, the method may include selectively oxidizing the halogen donor compound of the bioconjugation warhead at an electrochemically efficient voltage to generate a halogen radical. Also, the method may include contacting the halogen radical with the bioconjugation warhead at a constant voltage to form an electrophilic warhead. Also, the method may include reacting the electrophilic warhead with a coupling partner comprising a nucleophilic group of the biomolecule to conjugate the electrophilic warhead to the biomolecule.

In one example embodiment, the biomolecule may include, but is not limited to, nucleic acids, proteins, peptides, and the like. For instance, the biomolecule may be a nucleic acid. In another example embodiment, the biomolecule may be a protein. In another example embodiment, the biomolecule may be a peptide. In one example embodiment, the biomolecule may be bivalirudin. In another example embodiment, the biomolecule may be Angiotensin I. In another example embodiment, the biomolecule may be lysozyme. In another example embodiment, the biomolecule may be bovine serum albumin (BSA). In another example embodiment, the biomolecule may be tobacco mosaic virus (TMV).

As used herein, “bioconjugation warhead” refers to a reactive functional group or moiety that may be used in chemical reactions to attach biomolecules to other molecules. For instance, the bioconjugation warhead may target a specific site on a biomolecule.

In some example embodiments, the bioconjugation warhead may include, but is not limited to, a saturated or unsaturated, aromatic or non-aromatic, monocyclic, bicyclic or tricyclic carbon ring system. For instance, in some example embodiments, the bioconjugation warhead may include, but is not limited to, a non-aromatic monocyclic carbon ring system. In another example embodiment, the bioconjugation warhead may be a non-aromatic bicyclic carbon ring system.

In some example embodiments, the bioconjugation warhead may be a three-member ring system. For instance, in some example embodiments the three-member ring may include, but is not limited to, cyclopropanol (CPol) or a substituted CPol. The substituted CPol may further include, but is not limited to, hydroxyl, amino, alkyl, aryl, alkenyl, alkynyl, alkoxy, carboxy, benzyl, phenyl, nitro, thiol, thioalkoxy, halogen, or a combination thereof. In one example embodiment, the substituted CPol may include hydroxyl group. In another example embodiment, the substituted CPol may include an amino group. In another example embodiment, the substituted CPol may include an alkyl group. In another example embodiment, the substituted CPol may include an aryl group. In another example embodiment, the substituted CPol may include an alkenyl group. In another example embodiment, the substituted CPol may include an alkynyl group. In another example embodiment, the substituted CPol may include an alkoxy group. In another example embodiment, the substituted CPol may include a carboxy group. In another example embodiment, the substituted CPol may include a benzyl group. In another example embodiment, the substituted CPol may include a phenyl group. In another example embodiment, the substituted CPol may include a nitro group. In another example embodiment, the substituted CPol may include a thiol group. In another example embodiment, the substituted CPol may include a thioalkoxy group. In another example embodiment, the substituted CPol may include a halogen.

In some example embodiments, the CPol derivative may have the following Formula (I),

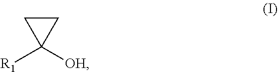

wherein R₁ may be selected from a group consisting of hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof. In one example embodiment, R₁ may include, but is not limited to, phenyl, toyl, benzyl, phenyl, methoxyphenyl, methyl, fluoromethyl, trifluoromethyl, propyl, isopropyl, or a combination thereof.

In one example embodiment, the CPol derivative may include, but is not limited to, phenylcyclopropanol, 1-(p-tolyl)cyclopropan-1-ol, benzylcylopropanol, pentylcyclopropanol, 1-(4-methoxyphenyl)cyclopropan-1-ol, 1-(4-(trifluoromethyl)phenyl)cyclopropan-1-ol, 1-(3-methoxyphenyl)cyclopropan-1-ol, 1-(3-(trifluoromethyl)phenyl)cyclopropan-1-ol, 1-isopropylcyclopropan-1-ol, 3-bromopropyl cyclopropanol, or a combination thereof.

In one example embodiment, the substituted CPol may be phenylcyclopropanol. For instance, phenylcyclopropanol may have the following Formula (I-1):

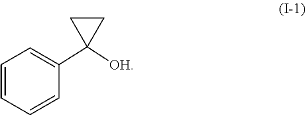

In one example embodiment, the substituted CPol may be 1-(p-tolyl)cyclopropan-1-ol. For instance, 1-(p-tolyl)cyclopropan-1-ol may have the following Formula (I-2):

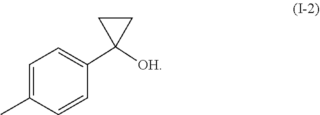

In one example embodiment, the substituted CPol may be benzylcylopropanol. For instance, benzylcylopropanol may have the following Formula (I-3):

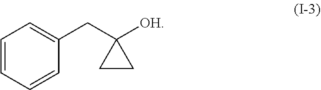 [SMILES: OC1(Cc2ccccc2)CC1]

In one example embodiment, the substituted CPol may be pentylcyclopropanol. For instance, pentylcyclopropanol may have the following Formula (I-4):

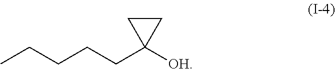

In one example embodiment, the substituted CPol may be 1-(4-methoxyphenyl)cyclopropan-1-ol. For instance, 1-(4-methoxyphenyl)cyclopropan-1-ol may have the following Formula (I-6):

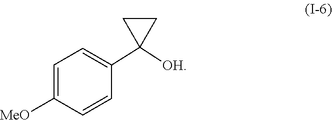 [SMILES: COc1ccc(C2(O)CC2)cc1]

In one example embodiment, the substituted CPol may be 1-(4-(trifluoromethyl)phenyl)cyclopropan-1-ol. For instance, 1-(4-(trifluoromethyl)phenyl)cyclopropan-1-ol may have the following Formula (I-7):

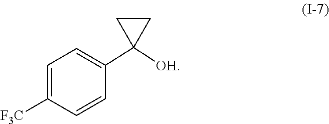 [SMILES: OC1(c2ccc(C(F)(F)F)cc2)CC1]

In one example embodiment, the substituted CPol may be 1-(3-methoxyphenyl)cyclopropan-1-ol. For instance, 1-(3-methoxyphenyl)cyclopropan-1-ol may have the following Formula (I-8):

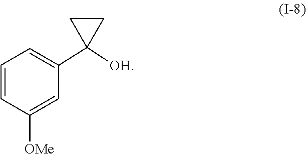 [SMILES: COc1cccc(C2(O)CC2)c1]

In one example embodiment, the substituted CPol may be 1-(3-(trifluoromethyl)phenyl)cyclopropan-1-ol. For instance, 1-(3-(trifluoromethyl)phenyl)cyclopropan-1-ol may have the following Formula (I-9):

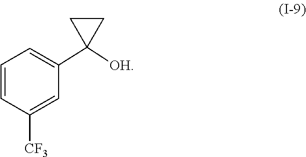 [SMILES: OC1(c2cccc(C(F)(F)F)c2)CC1]

In one example embodiment, the substituted CPol may be 1-isopropylcyclopropan-1-ol. For instance, 1-isopropylcyclopropan-1-ol may have the following Formula (I-10):

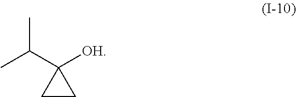

In one example embodiment, the substituted CPol may be 3-bromopropyl cyclopropanol. For instance, 3-bromopropyl cyclopropanol may have the following Formula (I-11):

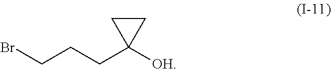

In some example embodiments, the bioconjugation warhead may include, but is not limited to, a halogen donor compound. As used herein, a “halogen donor” compound refers to a compound that can release or donate a halogen atom in a chemical reaction. For instance, a halogen donor compound may donate said halogen atom during an electrochemical process to form a compound which includes said halogen atom.

In some example embodiments, the halogen donor compound may include, but is not limited to, a metal. salt metal may include an alkali metal. For instance, the metal may include, but is not limited to, sodium, lithium, potassium, rubidium, aluminum, cesium, or francium. In one example embodiment, the metal may be sodium. In another example embodiment, the metal may be potassium. In another example embodiment, the metal may be lithium. In another example embodiment, the metal may be aluminum.

In some example embodiments, the halogen donor compound may be a sodium based compound. For instance, in some example embodiments, the sodium based compound may have the following Formula (II):

Y⁺—X⁻ (II),

wherein Y may be a metal; and X may be a halogen. In some example embodiments, the halogen donor compound may include, but is not limited to, sodium iodide, sodium bromide, lithium bromide, lithium iodide, potassium bromide, potassium iodine, tetraethylammonium bromide, tetraethylammonium chloride, or a combination thereof.

In one example embodiment, the halogen donor compound may comprise sodium iodide. For instance, sodium iodide may have the following Formula (IIa):

Na⁺I⁻ (IIa).

In one example embodiment, the halogen donor compound may comprise sodium bromide. For instance, sodium bromide may have the following Formula (IIb):

Na⁺Br⁻ (IIb).

Also, the present disclosure is directed to a method of forming a bioconjugated molecule. For instance, the bioconjugation warhead may be formed in an electrochemical system. For instance, the electrochemical system may include one or more electrochemical cells with a reference electrode and a working electrode. In one example embodiment, the reference electrode may be formed from any electrode material known in the art. For instance, the reference electrode may be formed from silver, silver chloride, or alloys, or a combination thereof. Nonetheless, the reference electrode may be selected from a material that is capable of delivering a current, such as a direct current (DC) or alternating current (AC) voltage signal. For instance, as generally known in the art, the reference electrode is selected and formed so as to form a stable voltage at the interface during the time of the measurement. An Ag/AgCl reference electrode is therefore often used for that reason.

In one example embodiment, the anode may include, but is not limited to, zinc, iron, chromium, nickel, lead, titanium, copper, tin, silver, lead(IV) oxide, manganese(IV) oxide, sulfur, Prussian blue, Prussian blue derivatives, transition metal analogs of Prussian blue, carbon fiber, graphite, carbon felt, conductive carbon black, as well as other conductive forms of carbon. For instance, the anode may include one or more layers including, without limitation, a separator, e.g., a porous carbon paper, carbon cloth, carbon felt, or metal cloth (e.g., a porous film made of fiber-type metal or a metal film formed on the surface of a polymer fiber cloth), a conductive substrate appropriate for the electrode electrolyte solution of the cell (e.g., graphite), and a current collector (e.g., gold-plated copper). In one example embodiment, the cathode may include, but is not limited to, nickel, iron, copper, ruthenium, platinum, palladium, rhodium, iridium, rhenium, silver, gold, or a combination thereof.

In one example embodiment, the electrochemical system may further include an electrolyte solution. For instance, the electrode electrolyte solution may be an aqueous solution that includes a redox component. The redox component may be oxidized at a first electrode and reduced at a second electrode as the electrode electrolyte solution is cycled through the electrochemical cell under a voltage potential established between the first and second electrodes.

In one example embodiment, the electrode electrolyte solution may include, but is not limited to, at least one redox component that is capable of a reversible redox reaction under the voltage potential of the electrochemical cell, providing a reversible redox pair. By way of example, and without limitation, the redox component can include one or more of, and without limitation to, an ion of titanium (titanium(III), titanium(IV)), vanadium (vanadium(II), vanadium(III), vanadium(IV), vanadium(V)), chromium (chromium(II), chromium(III), chromium(VI)), manganese (manganese(II), manganese(III), manganese(VI), manganese(VII)), iron (iron(II), iron(III), iron(VI)), cobalt (cobalt(II), cobalt(III)), nickel (nickel(II)), copper (copper(I), copper(II)), zinc (zinc(II)), ruthenium (ruthenium(II), ruthenium(III)), tin (tin(II), tin(IV)), cerium (cerium(III), cerium(IV)), tungsten (tungsten(IV), tungsten(V)), osmium (osmium(II), osmium(III)), lead (lead II), zincate, aluminate, chlorine, chloride, bromine, bromide, tribromide, iodine, iodide, triiodide, polyhalide, halide oxyanion, sulfide, polysulfide, sulfur oxyanion, ferrocyanide, ferricyanide, a quinone derivative, an alloxazine derivative, a flavin derivative, a viologen derivative, a metallocene derivative (e.g., a ferrocene derivative), a nitroxide radical derivative, a N,N-dialkyl-N-oxoammonium derivative, a nitronyl nitroxide radical derivative, and/or polymers incorporating complexed or covalently bound components of any of the aforementioned materials.

By way of example, a redox component can be present in the electrode electrolyte solution as a salt such as, and without limitation to, cerium chloride, germanium chloride, vanadium chloride, europium chloride, or ferrous chloride.

A redox pair of the electrode electrolyte solution can be an anion-based pair or a cation-based pair. For instance, a solution can include an anion-based redox component such as an aluminum-based Al(OH)₄⁻/Al redox pair, a zinc-based Zn(OH)₄²⁻/Zn redox pair, a sulfur-based S₄²⁻/S₂²⁻ redox pair, a cobalt-based Co(CN)₆³⁻/Co(CN)₆⁴⁻, or a bromine Br₃⁻/Br⁻ redox pair. Examples of cation-based redox components can include, without limitation, vanadium based redox pairs such as vanadium-based VO⁺/VO²⁺ or V³⁺/V²⁺ redox pairs, a zinc-based Zn²⁺/Zn redox pair, a cerium-based Ce⁴⁺/Ce³⁺ redox pair, a chromium-based Cr³⁺/Cr²⁺ redox pair, an iron-based Fe³⁺/Fe²⁺ redox pair, a cobalt-based Co³⁺/Co²⁺ redox pair, etc.

In addition to one or more compounds capable of providing the desired redox pair, the electrode electrolyte solution can include one or more solutes and solvents, pH buffers, etc. as are generally known in the art. For instance, a solution can include a pH buffer that may or may not be redox-active under typical operating conditions. In one example embodiment, the pH of the electrode electrolyte solution can be matched to the pH of the salt solution that includes the targeted ion. As such, the electrode electrolyte solution can be approximately neutral (e.g., pH from about 5 to about 9), acidic (e.g., pH less than about 5), or alkaline (e.g., pH greater than about 9), depending upon the characteristics of the system and the particular method.

In one example embodiment, the electrochemical system may further include one or more ion exchange membranes. For instance, the ion exchange membranes may be anion exchange membranes or cation exchange membranes, depending on the nature of the ion targeted for separation by the system and the redox component of the electrode electrolyte solution. While both ion exchange membranes will be either anion exchange membranes or cation exchange membranes, they can be of the same composition or different, as desired.

The ion exchange membranes may be water permeable. The ion exchange membranes may include, but are not limited to, commercially available membranes and membranes with chemical modifications. Non-limiting examples of such modifications are: (i) perfluorinated films with fixed pyridine or sulfonic groups; (ii) polyetherketones; (iii) polysulfonones; (iv) polyphenylene oxides; (v) polystyrene; (vi) styrene-divinyl benzene; (vii) polystyrene/acrylic based fabrics with sulfonate and quaternary ammonium cations; (viii) polyfluorinated sulfuric acid polymers; or (ix) resin-polyvinylidenedifluoride fabrics.

An anion exchange membrane as may be incorporated in an electrochemical cell can include, but is not limited to, a membrane that allows passage of anions and does not allow passage of cations. In one example embodiment, an anion exchange membrane can be a negative-valence selective membrane that allows passage of anions having a negative charge greater than a cut-off value while not allowing passage of anions having a negative charge less than the cut-off value.

Examples of anion exchange membranes as may be incorporated in an electrochemical cell can include, without limitation, those marketed under the tradename NEOSEPTA® and being of the grade AM-1, AMX, ACS and ACS-3, available from Tokuyama Corp., Tokyo, Japan; those marketed under the tradename FUMASEP® FAB, available from FuMA-Tech GmbH, Germany; and those marketed under the tradename ZIRFON®, available from Agfa Corp.

A cation exchange membrane as may be incorporated in an electrochemical cell can include a membrane that allows passage of cations and does not allow passage of anions. In one example embodiment, a cation exchange membrane can be a positive-valence selective membrane that allows passage of cations having a positive charge greater than a cut-off value while not allowing passage of cations having a positive charge less than the cut-off value.

Examples of cation exchange membranes as may be incorporated in an electrochemical cell can include, without limitation, those marketed under the tradename NEOSEPTA® and being of grade CM-1, CMX, CMS and CIMS, available from Tokuyama Corp., Tokyo, Japan; a sulfonated-tetrafluoroethylene-based fluoropolymer-copolymer commercially available under the tradename NAFION®, available from E. I. du Pont de Nemours and Company.

In one example embodiment, the electrochemical cell may be an undivided electrochemical cell, meaning that the anode and cathode are in the same electrochemical chamber. In another example embodiment, the electrochemical cell may be a divided electrochemical cell, meaning that the anode and cathode are in different electrochemical chamber separated by an ion exchange membrane.

To form the CPol derivative, the bioconjugation warhead and the halogen donor compound may undergo an electrochemical reaction in the electrochemical cell, which results in a covalent linkage between the bioconjugation warhead and the halogen atom. For instance, the bioconjugation warhead and the halogen donor compound may be mixed in the same electrode electrolyte solution in the electrochemical system. In one example embodiment, the bioconjugation warhead and the halogen donor compound may be present in the electrode electrolyte solution at a molar ratio of from about 1:10 to about 10:1, such as from about 1:5 to about 5:1, such as from about 1:3 to about 3:1, such as from about 1:2 to about 2:1, or any range therebetween. For instance, the bioconjugation warhead and the halogen donor compound may be present in the electrode electrolyte solution at a molar ratio of from about 1:5 to about 5:1. In another example embodiment, the bioconjugation warhead and the halogen donor compound may be present in the electrode electrolyte solution at a molar ratio of from about 1:2 to about 2:1.

Advantageously, the oxidation potential of the halogen donor compound is lower than the oxidation potential of the bioconjugation warhead. For instance, the oxidation potential of the halogen donor compound is at least about 10% lower than the oxidation potential of the bioconjugation warhead, such as at least about 15% lower, such as at least about 20% lower.

In some example embodiments, the halogen atom may be selectively oxidized to form a halogen radical under mild electrochemical conditions. For instance, an electrochemically efficient voltage may be applied to the electrochemical cell to selectively oxidize the halogen donor compound without oxidizing the bioconjugation warhead. As used herein, “electrochemically efficient voltage” refers to a low voltage amount that generates a halogen radical. For instance, to selectively oxidize the halogen donor compound, an electrochemically efficient voltage of from about 0.1 V to about 2.0 V may be applied to the electrochemical cell, such as from about 0.3 V to about 1.5 V, such as from about 0.5 V to about 1.0 V. In one example embodiment, an electrochemically efficient voltage of from about 0.3 V to about 1.5 V may be applied to the electrochemical cell to selectively oxidize the halogen donor compound. Selective oxidation of the halogen donor compound forms a halogen radical.

In some example embodiments, after formation of the halogen radical, a constant voltage may continue to be applied to the electrochemical cell, which facilitates ring opening of the bioconjugation warhead. As such, the halogen radical may initiate opening the substituted CPol ring via β-scission process, thereby forming the CPol derivative.

In some example embodiments, the resulting CPol derivative may act as an electrophilic warhead. For instance, the electrophilic warhead may include a β-substituted ketone. In one example embodiment, the electrophilic warhead have the following Formula I-#:

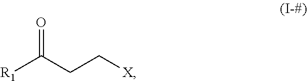

wherein R₁ may be selected from a group consisting of hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, or a combination thereof: and
wherein X may be a halogen.

In one example embodiment, the electrophilic warhead may include, but is not limited to, 3-iodo-1-phenylpropan-1-one, 3-iodo-1-(p-tolyl)propan-1-one, 4-iodo-1-phenylbutan-2-one, 1-iodooctan-3-one, 1-iodopentan-3-one, 3-iodo-1-(4-methoxyphenyl)propan-1-one, 3-iodo-1-(4-(trifluoromethyl)phenyl)propan-1-one, 3-iodo-1-(3-methoxyphenyl)propan-1-one, 3-iodo-1-(3-(trifluoromethyl)phenyl)propan-1-one, 1-iodo-4-methylpentan-3-one, N-(2-hydroxyethyl)-6-iodo-N,N-dimethyl-4-oxohexan-1-aminium bromide, or a combination thereof. In another example embodiment, the electrophilic warhead may include, but is not limited to, 3-bromo-1-phenylpropan-1-one, 3-bromo-1-(p-tolyl)propan-1-one, 4-bromo-1-phenylbutan-2-one, 1-bromooctan-3-one, 1-bromopentan-3-one, 3-bromo-1-(4-methoxyphenyl)propan-1-one, 3-bromo-1-(4-(trifluoromethyl)phenyl)propan-1-one, 3-bromo-1-(3-methoxyphenyl)propan-1-one, 3-bromo-1-(3-(trifluoromethyl)phenyl)propan-1-one, 1-bromo-4-methylpentan-3-one, N-(2-hydroxyethyl)-6-bromo-N,N-dimethyl-4-oxohexan-1-aminium bromide, or a combination thereof.

In one example embodiment, the electrophilic warhead may be 3-iodo-1-phenylpropan-1-one. For instance, 3-iodo-1-phenylpropan-1-one may have the following Formula (I-1a):

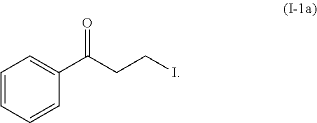 [SMILES: O=C(CCI)c1ccccc1]

In one example embodiment, the electrophilic warhead may be 3-iodo-1-(p-tolyl)propan-1-one. For instance, 3-iodo-1-(p-tolyl)propan-1-one may have the following Formula (I-2a):

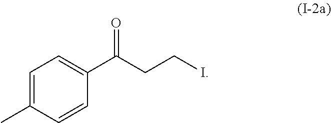

In one example embodiment, the electrophilic warhead may be 4-iodo-1-phenylbutan-2-one. For instance, 4-iodo-1-phenylbutan-2-one may have the following Formula (I-3a):

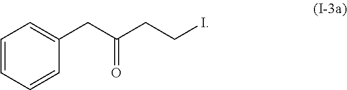 [SMILES: O=C(CCI)Cc1ccccc1]

In one example embodiment, the electrophilic warhead may be 1-iodooctan-3-one. For instance, 1-iodooctan-3-one may have the following Formula (I-4a):

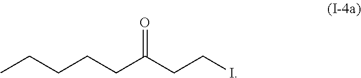

In one example embodiment, the electrophilic warhead may be 1-iodopentan-3-one. For instance, 1-iodopentan-3-one may have the following Formula (I-5a):

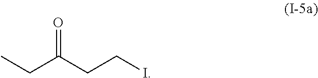

In one example embodiment, the electrophilic warhead may be 3-iodo-1-(4-methoxyphenyl)propan-1-one. For instance, 3-iodo-1-(4-methoxyphenyl)propan-1-one may have the following Formula (I-6a):

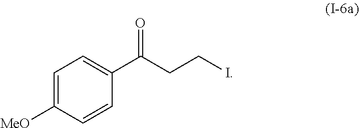 [SMILES: COc1ccc(C(=O)CCI)cc1]

In one example embodiment, the electrophilic warhead may be 3-iodo-1-(4-(trifluoromethyl)phenyl)propan-1-one. For instance, 3-iodo-1-(4-(trifluoromethyl)phenyl)propan-1-one may have the following Formula (I-7a):

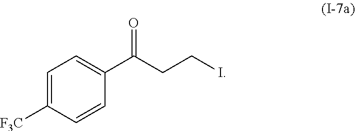 [SMILES: O=C(CCI)c1ccc(C(F)(F)F)cc1]

In one example embodiment, the electrophilic warhead may be 3-iodo-1-(3-methoxyphenyl)propan-1-one. For instance, 3-iodo-1-(3-methoxyphenyl)propan-1-one may have the following Formula (I-8a):

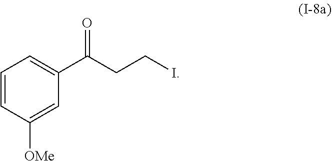 [SMILES: COc1cccc(C(=O)CCI)c1]

In one example embodiment, the electrophilic warhead may be 3-iodo-1-(3-(trifluoromethyl)phenyl)propan-1-one. For instance, 3-iodo-1-(3-(trifluoromethyl)phenyl)propan-1-one may have the following Formula (I-9a):

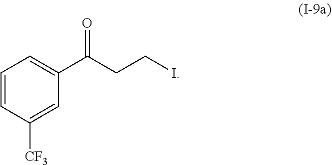 [SMILES: O=C(CCI)c1cccc(C(F)(F)F)c1]

In one example embodiment, the electrophilic warhead may be 1-iodo-4-methylpentan-3-one. For instance, 1-iodo-4-methylpentan-3-one may have the following Formula (I-10a):

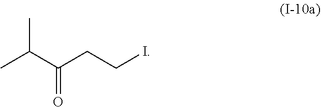

In one example embodiment, the electrophilic warhead may be N-(2-hydroxyethyl)-6-iodo-N,N-dimethyl-4-oxohexan-1-aminium bromide. For instance, N-(2-hydroxyethyl)-6-iodo-N,N-dimethyl-4-oxohexan-1-aminium bromide may have the following Formula (I-11a):

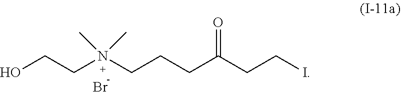

In one example embodiment, the electrophilic warhead may be 3-bromo-1-phenylpropan-1-one. For instance, 3-bromo-1-phenylpropan-1-one may have the following Formula (I-1b):

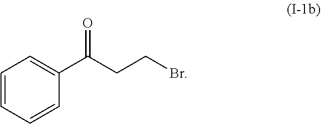 [SMILES: O=C(CCBr)c1ccccc1]

In one example embodiment, the electrophilic warhead may be 3-bromo-1-(p-tolyl)propan-1-one. For instance, 3-bromo-1-(p-tolyl)propan-1-one may have the following Formula (I-2b):

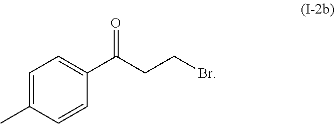

In one example embodiment, the electrophilic warhead may be 4-bromo-1-phenylbutan-2-one. For instance, 4-bromo-1-phenylbutan-2-one may have the following Formula (I-3b):

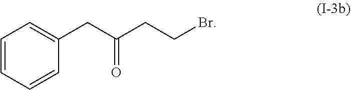 [SMILES: O=C(CCBr)Cc1ccccc1]

In one example embodiment, the electrophilic warhead may be 1-bromooctan-3-one. For instance, 1-bromooctan-3-one may have the following Formula (I-4b):

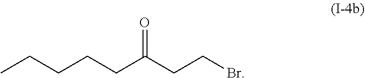

In one example embodiment, the electrophilic warhead may be 1-bromopentan-3-one. For instance, 1-bromopentan-3-one may have the following Formula (I-5b):

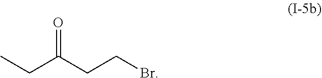

In one example embodiment, the electrophilic warhead may be 3-bromo-1-(4-(trifluoromethyl)phenyl)propan-1-one. For instance, 3-bromo-1-(4-(trifluoromethyl)phenyl)propan-1-one may have the following Formula (I-7b):

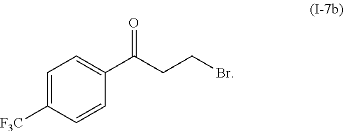 [SMILES: O=C(CCBr)c1ccc(C(F)(F)F)cc1]

In one example embodiment, the electrophilic warhead may be 3-bromo-1-(3-(trifluoromethyl)phenyl)propan-1-one. For instance, 3-bromo-1-(3-(trifluoromethyl)phenyl)propan-1-one may have the following Formula (I-9b):

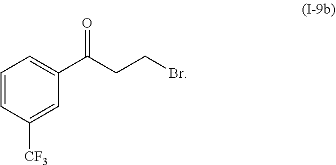 [SMILES: O=C(CCBr)c1cccc(C(F)(F)F)c1]

In one example embodiment, the electrophilic warhead may be 1-bromo-4-methylpentan-3-one. For instance, 1-bromo-4-methylpentan-3-one may have the following Formula (I-10b):

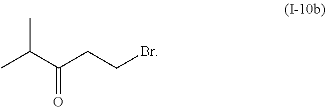

In some example embodiments, the electrophilic warhead may be selectively conjugated to a biomolecule via an electrochemical reaction. For instance, the electrophilic warhead may be conjugated to the N-terminus of a biomolecule. In another example embodiment, the electrophilic warhead may be conjugated to the C-terminus of a biomolecule. For instance, the electrophilic warhead may be conjugated to a biomolecule as a biorthogonal tag. In one example embodiment, the electrophilic warhead disclosed herein may be conjugated resulting in a conjugated biomolecule. To conjugate the electrophilic warhead to a biomolecule, an electrochemically efficient voltage of from about 0.1 V to about 2.0 V may be applied to the electrochemical cell, such as from about 0.3 V to about 1.5 V, such as from about 0.5 V to about 1.0 V, or any range therebetween.

The electrophilic warhead disclosed herein may be conjugated to any compatible biomolecule to be labeled. In some example embodiments, the biomolecule may have an average molecular weight (Mw) of at least about 1,000 g/mol, such as at least about 2,000 g/mol, such as at least about 10,000 g/mol, such as at least about 20,000 g/mol, such as at least about 50,000 g/mol, such as at least about 100,000 g/mol, such as at least about 250,000 g/mol, such as at least about 500,000 g/mol. In one example embodiment, the biomolecule may have an average Mw in the range of about 1,000 g/mol to about 30,000 g/mol, or any range therebetween.

The electrophilic warhead disclosed herein may be a valuable tool for real-time cell imaging or various other bioconjugation applications. Advantageously, the conjugated biomolecule may be utilized in cell labeling. For instance, the conjugated biomolecule may be incorporated into mammalian cells via the biosynthetic process.

In one example embodiment, the bioconjugation warhead may be useful for imaging live cells in real time. For instance, the bioconjugation warhead disclosed herein may be incorporated into a cell via incubating the live cells with the bioconjugation warhead for a sufficient time to allow for metabolic incorporation of the bioconjugation warhead. As such, incubation of the live cells with the bioconjugation warhead may electrochemically promote the formation of the conjugated biomolecule within the live cells. To image the live cells, the cell may be electrochemically reacted with the conjugated biomolecule and a dye. For instance, the dye may be a fluorescent dye. In one example embodiment, for instance, the fluorescent dye may be fluorescein thiosemicarbazide.

The preceding description is exemplary in nature and is not intended to limit the scope, applicability or configuration of the disclosure in any way. Various changes to the described embodiments may be made in the function and arrangement of the elements described herein without departing from the scope of the disclosure.

Here and throughout the specification and claims, range limitations arc combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Materials and Methods

### Virus Purification

To purify wild-type plant virus tobacco mosaic virus (TMV), frozen TMV leaves were crushed and blended. Approximately 3 volumes of phosphate buffer were added to the crushed leaves with an addition of 0.2-0.3% 2-mercaptoethanol. The leaves were blended for 2 min at a low setting and then switched to the highest setting for an additional 3 min. The blended plant sap was filtered through two layers of cheesecloth and then flow through was centrifuged at 10500 rpm (Sorvall SLA 1500) at 4° C. for 20 min. The resulting supernatant was then pooled together and mixed with n-butanol/chloroform at a ratio of 2:1:1 (plant sap/n-butanol/chloroform). The homogenate was stirred for 30 min on ice and then centrifuged at 6000 rpm (SLA 4000) for 10 min at 4° C. The upper aqueous layer was transferred to a beaker, and the virus was precipitated by adding 0.2 M NaCl and 8% (wt/vol) PEG-8000. The mixture was stirred on ice for 60 min and centrifuged at 12000 rpm (Sorvall SLA1500) for 20 min. The white precipitant was then resuspended with 10 mM K phos buffer (pH 7.8). The solution was centrifuged at 9,500 rpm for 15 min at 4° C. to remove excess PEG. The supernatant was transferred to ultracentrifuge tubes (Beckman 50.2 Ti) and the virus was pelleted at 42,000 rpm at 4° C. for 3 h. The virus pellet was resuspended in 100 mM K phos buffer (pH 7.8) overnight at 4° C. UV absorbance was measured at 260 and 280 nm wavelengths to determine the concentration of the virus. The virus concentration of 21.6 mg/mL has an absorbance of 0.324 at 260 nm and exhibits a characteristic 260/280 ratio of 1.2. The purified virus solutions were stored at 4° C.

### MALDI-TOF MS Analysis

Mass spectra were generated with a MALDI-TOF mass spectrometer (rapifleX MALDI Tissuetyper from BRUKER) equipped with a solid-state smart beam and operated in a linear delayed extraction positive ion mode. The samples of conjugated peptide were concentrated using IMCStips (1 mL, IMCS company) and eluted with 70% acetonitrile/0.1% trifluoracetic acid (TFA) following the manufacturer protocol. Of note, the samples of conjugated proteins and viruses were denatured by incubating with guanidinium chloride (8 M) for 5 min at RT, followed by the removal of salts and elution using IMCStips. The resultant samples were spotted on a MALDI plate. These spots were analyzed by MALDI-TOF MS spectrometer with the matrix (Vₛₐₘₚₗₑ: Vₘₐₜᵣᵢₓ=1:1). Matrix of sinapic acid in 50% acetonitrile/0.1% TFA was used for protein measurements, and the matrix of a-Cyano-4-hydroxycinnamic acid in 50% acetonitrile/0.1% TFA was used for peptide measurements.

### Cell Culture

Vero cells (from CDC) were maintained in high glucose DMEM containing 2 mM L-glutamine, 1 mM sodium pyruvate, 100 U/mL penicillin-streptomycin (from HyClone), and supplemented with 10% fetal bovine serum (FBS, from Atlanta Biologicals). Cell cultures were incubated in a 5% CO₂ incubator at 37° C.

### Cell Viability Assay

The in vitro cytotoxicity test of CPol-choline analog or other chemicals on Vero cells was determined by CellTiter Blue (CTB) cell viability assay (Promega). Briefly, Vero cells were cultured in 96-well plates at a density of 12.5×10³ cells per well one day prior experiment. On the following day, the media was replaced by fresh 10% FBS DMEM containing the choline analogs at the concentrations of 0.2, 0.4, 0.8, 1, and 5 mM and incubated for 24 h at 37° C. The cells without the compounds were used as controls. NaI and I₂ were also tested at the concentrations varied from 0.01 to 10,000 μM. The experiments were performed in quadruplicate. After 24 h of incubation, the media in each well was replaced by prewarmed media containing 10% CTB reagent and incubated for 2 h at 37° C. and 5% CO₂. The medium containing CTB without cells was used as a negative control. Fluorescence intensity was then measured at 560/590 nm (Ex/Em) using SpectraMax M2 Multi-Mode microplate reader (Molecular Devices) to indicate cell viability.

### Extraction of Lipids From Vero/HeLa/NIH-3T3 Cells

Three million cells (Vero, HeLa, or NIH-3T3) were seeded into a 10-cm cell culture dish and allowed attachment for 24 h in a complete medium (DMEM with 10% FBS) in a CO₂ incubator. When cells reached 70-80% confluency, choline analog (PCPol-Cho or ECPol-Cho) at a final concentration of 5 mM was added into cells and co-cultured for another 24 h. The adhered cells were washed with 5 mL ice-cold DPBS (Dulbecco's Phosphate-Buffered Saline, CORNING), and were scratched into ice-cold PBS (5 mL), pooled in a centrifuge tube, and centrifuged at 1,000 rpm for 5 min at room temperature. The supernatant was then aspirated off and the lipids were extracted from the resultant pellet using the modified Bligh Dyer method. Briefly, the cell pellet was treated with a mixture of chloroform and methanol (1 mL of a 1:2 v/v solution), vortexed for 1 min, followed by addition of chloroform (0.5 mL) and 1 M NaCl (0.5 mL), and again vortexed for 1 min. Phase separation was achieved by centrifuging the mixture at 3,220 g at 4° C. for 30 min. The lower lipid-enriched organic layer was separated and dried using nitrogen at room temperature. For all LC-MS/MS analyses, the dried lipids were dissolved in a mixture of chloroform and methanol (200 μL of a 1:1 v/v solution). For all control experiments, the procedure described above was followed without administration of any choline derivatives.

### Cell Labeling of CPol-Choline Analog in Vero Cells

Vero cells were cultured in a 100-mm cell culture dish and allowed attachment overnight in a complete medium (DMEM with 10% FBS). When cells reached 60-70% confluency, CPol-choline analog at a final concentration of 5 mM was added into Vero cells and co-cultured for 24 h. The labeled Vero cells were harvested into 5 mL Dulbecco's Phosphate-Buffered Saline (DPBS 1×, CORNING). The cell numbers were counted using a hemocytometer. The cell concentration at 6×10⁶ cells was used in the experiment. Subsequently, the Vero cell suspension was treated with NaI (100 μM) and low cell potential (0.7 V) under divided H-cell for 10 min. Then, the treated cells were seeded to a 24-well plate at the concentration of 2×10⁴ cells per well for confocal imaging and to a 96-well plate at the concentration of 7.5'10⁴ cells per well for intracellular fluorescent measurement. The cells were maintained in the medium of 2% FBS DMEM without sodium pyruvate and penicillin-streptomycin overnight. On the following day, the conjugated Vero cells were washed with DPBS three times. For the fluorescence imaging, the cells in a 24-well plate were fixed with 70% ethanol at −20° C. for 10 min, washed with DPBS three times, and then reacted with 50 μM FTSCA for 1 h at room temperature. After removing the unbound FTSCA, the cells were imaged by a fluorescence microscope (Olympus IX81, Shinjuku-ku, Tokyo, Japan). For intracellular fluorescent measurement, the cells in a 96-well plate were co-culture with 50 μM FTSCA in the medium of 2% FBS DMEM at 37° C. incubator for 1 h. After washing with DPBS, the plate was read using a plate reader (Tecan Infinite M200 microplate reader) with an excitation wavelength of 491 nm and emission cutoff at 520 nm.

### Example 1

### Cpol Derivatives Synthesis

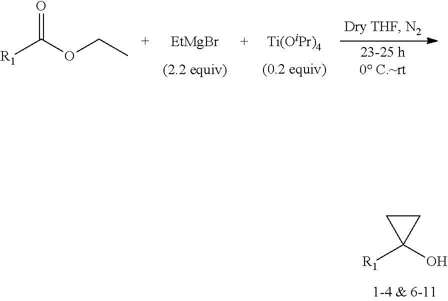

Ethylmagnesium bromide (10.6 mmol, 10.6 mL, 1 M in THF) was added dropwise over 1 h at 4° C. to a solution of ester (5 mmol) and titanium tetraisopropoxide (1 mmol, 0.3 mL) in THF (15 mL) under N₂ protection. The mixture was then stirred at room temperature for 23-25 h. Then the reaction was quenched by the addition of water (about 15 mL) under an ice bath while stirring. After that, the mixture was filtered by vacuum filtration obtaining a liquid solution. The solution then was extracted with anhydrous diethyl ether (3×20 mL) and saturated NH₄Cl (1×40 mL). The organic layers were combined and dried with Na₂SO₄, and the crude mixture was purified by column chromatography on silica gel (Hexane: EtOAc=20:1) to yield the desired cyclopropanol corresponding to Formulas I-1 through I4 and I-6 through I-11 herein.

Characterization data of each CPol derivative is described below:

Phenylcyclopropanol (I-1). The yellow oil was obtained in a 39% isolated yield. ¹H NMR (300 MHz, DMSO) δ 7.32-7.20 (m, 4H), 7.19-7.11 (m, 1H), 5.91 (s, 1H), 1.10 (m, 2H), 0.93 (m, 2H). ¹³C NMR (75 MHz, DMSO) δ 146.0; 127.9; 125.3; 123.8; 54.3; 18.2.

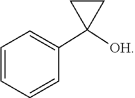

1-(p-tolyl)cyclopropan-1-ol (I-2). The yellow oil was obtained in a 42.5% isolated yield. ¹H NMR (300 MHz, DMSO) δ 7.15-7.06 (m, 4H), 5.83 (s, 1H), 2.26 (s, 3H), 1.05 (m, 2H), 0.88 (m, 4.7 Hz, 2H). ¹³C NMR (75 MHz, DMSO) δ 142.9; 134.2; 128.4; 123.9; 54.2; 20.5; 17.8.

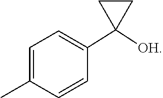

Benzylcylopropanol (I-3). Light yellow oil was obtained in 60.8% isolated yield. ¹H NMR (300 MHz, DMSO) δ 7.33-7.14 (m, 5H), 5.17 (s, 1H), 2.81 (s, 2H), 0.64-0.55 (m, 2H), 0.54-0.45 (m, 2H). ¹³C NMR (75 MHz, DMSO) δ 139.7; 129.5; 127.8; 125.8; 54.5; 43.7; 12.5.

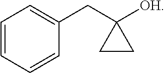 [SMILES: OC1(Cc2ccccc2)CC1]

Pentylcyclopropanol (I-4). Colorless oil was obtained in a 76.3% isolated yield. ¹H NMR (300 MHz, DMSO) δ 4.90 (s, 1H), 1.48-1.36 (m, 4H), 1.28 (m, 4H), 0.86 (t, J=6.9 Hz, 3H), 0.50 (m, 2H), 0.28 (m, 2H). ¹³C NMR (75 MHz, DMSO) δ 54.2; 38.6; 31.9; 25.9; 22.7; 14.4; 13.2.

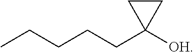

1-(4-methoxyphenyl)cyclopropan-1-ol (I-6). The yellow solid was obtained in 47.4% isolated yield. ¹H NMR (300 MHz, DMSO) δ 7.23-7.17 (m, 2H), 6.90-6.85 (m, 2H), 5.84 (s, 1H), 3.75 (s, 3H), 1.05 (m, 2H), 0.89 (m, 2H). ¹³C NMR (75 MHz, DMSO) δ 157.3; 137.8; 125.3; 113.3; 55.0; 54.2; 17.27.

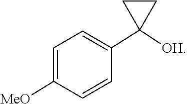 [SMILES: COc1ccc(C2(O)CC2)cc1]

1-(4-(trifluoromethyl)phenyl)cyclopropan-1-ol (I-7). Yellow crystal was obtained in 54.6% isolated yield. ¹H NMR (400 MHz, CDCl₃) δ 7.58 (d, J=8.3 Hz, 2H), 7.38 (d, J=8.2 Hz, 2H), 2.42 (s, 1H), 1.36 (m, 2H), 1.11 (m, 2H). ¹³C NMR (101 MHz, CDCl₃) δ 148.9; 126.4; 125.8; 125.4 (q, J=3.8 Hz); 124.3; 56.4; 19.2.

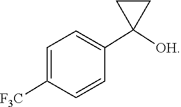 [SMILES: OC1(c2ccc(C(F)(F)F)cc2)CC1]

1-(3-methoxyphenyl)cyclopropan-1-ol (I-8). The yellow oil was obtained in 35.8% isolated yield. ¹H NMR (300 MHz, DMSO) δ 7.18 (t, J=7.9 Hz, 1H), 6.87-6.82 (m, 1H), 6.77-6.69 (m, 2H), 5.90 (s, 1H), 3.73 (s, 3H), 1.08 (m, 2H), 0.93 (m, 2H). ¹³C NMR (101 MHz, DMSO) δ 159.2; 147.9; 129.0; 115.9; 110.7; 109.9; 54.9; 54.3; 18.3.

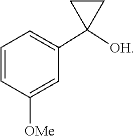 [SMILES: COc1cccc(C2(O)CC2)c1]

1-(3-(trifluoromethyl)phenyl)cyclopropan-1-ol (I-9). Yellow crystal was obtained in a 33.6% isolated yield. ¹H NMR (300 MHz, CDCl₃) δ 7.58 (s, 1H), 7.50-7.39 (m, 3H), 2.59 (s, 1H), 1.34 (m, 2H), 1.09 (m, 2H). ¹³C NMR (75 MHz, CDCl₃) δ 145.7; 131.1; 128.9; 127.5; 123.2 (q, J=3.8 Hz), 122.6; 121.3 (q, J=3.9 Hz), 56.4; 18.7.

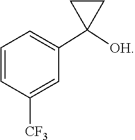 [SMILES: OC1(c2cccc(C(F)(F)F)c2)CC1]

1-isopropylcyclopropan-1-ol (I-10). The yellow oil was obtained in 16% isolated yield. ¹H NMR (400 MHz, CDCl₃) δ 1.73 (s, 1H), 1.30 (m, 1H), 1.02 (d, J=6.8 Hz, 6H), 0.71 (m, 2H), 0.44 (m, 2H). ¹³C NMR (101 MHz, CDCl₃) δ 60.2; 35.2; 18.5; 13.3.

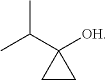

3-bromopropyl cyclopropanol (I-11). The yellow oil was obtained in a 32.6% isolated yield. ¹H NMR (300 MHz, DMSO) δ 4.83 (s, 1H), 3.58 (t, J=6.8 Hz, 2H), 2.00 (m, 2H), 1.54 (t, 6.4 Hz, 2H), 0.53 (m, 2H), 0.33 (m, 2H).

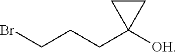

### Example 2

### E-Chem Promoted Ring-Opening Reactions of CPol Derivative

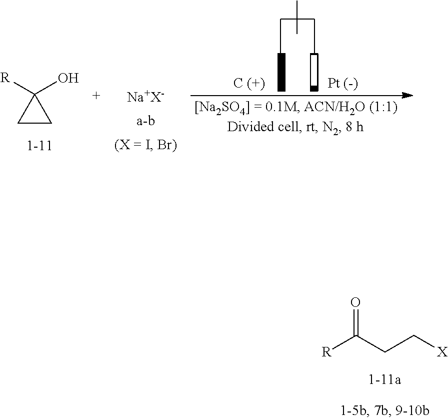

In a divided H-cell equipped with a stir bar, CPol derivatives (0.2 mmol) and NaI (0.36 mmol) or NaBr (0.36 mmol) were mixed in 6 mL acetonitrile/water (1:1) solvents containing 0.1 M Na₂SO₄ electrolyte. The H-cell was equipped with a carbon sheet as an anode, Pt sheet as the cathode, and Ag/AgCl as a reference electrode. Then, the H-cell was exposed to a nitrogen atmosphere for 10 minutes. The reaction mixture was stirred and electrolyzed at a constant potential (0.7 V for NaI, 1.2 and 1.5 V for NaBr) for 8 h at room temperature. After stopping the reaction, the reaction mixture was extracted with EA (3×10 mL) and saturated NH₄Cl (1×15 mL). The organic layer was dried over Na₂SO₄ and concentrated in a vacuum.

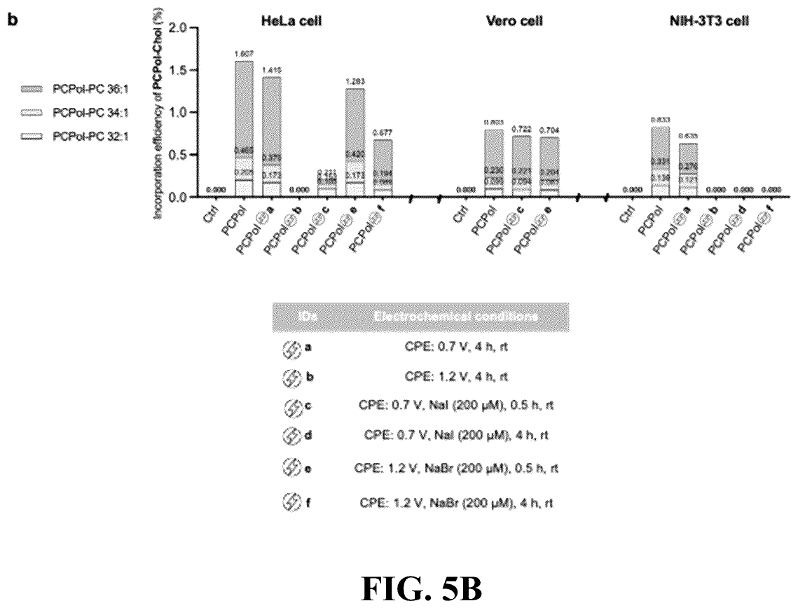

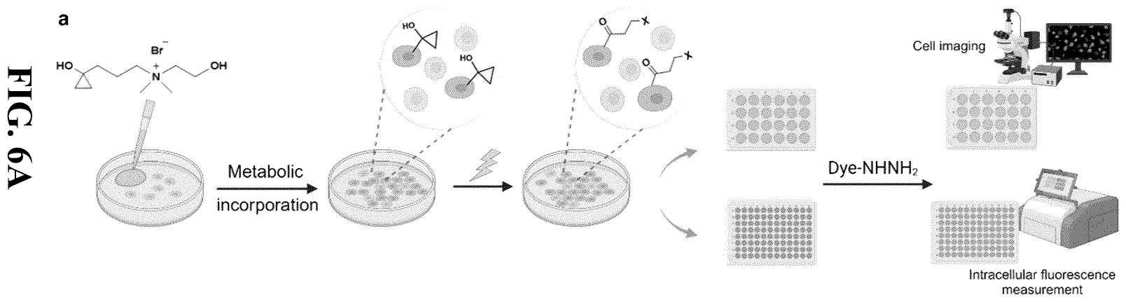

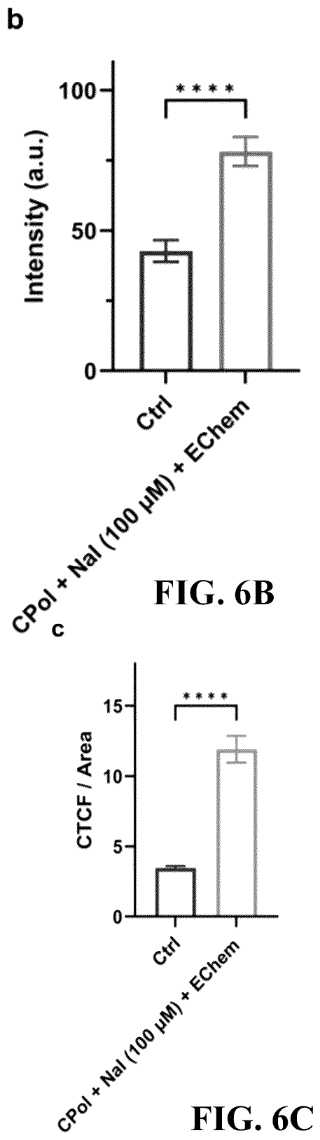

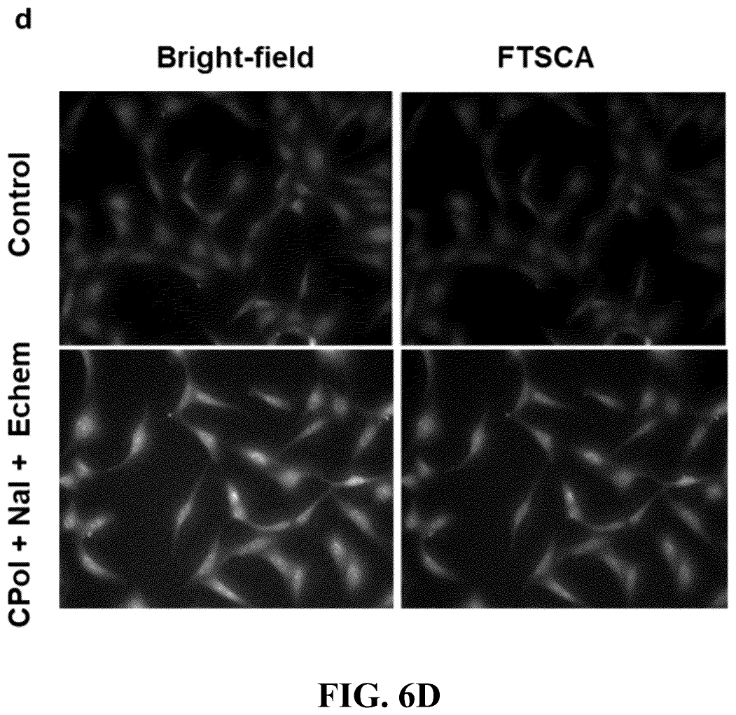

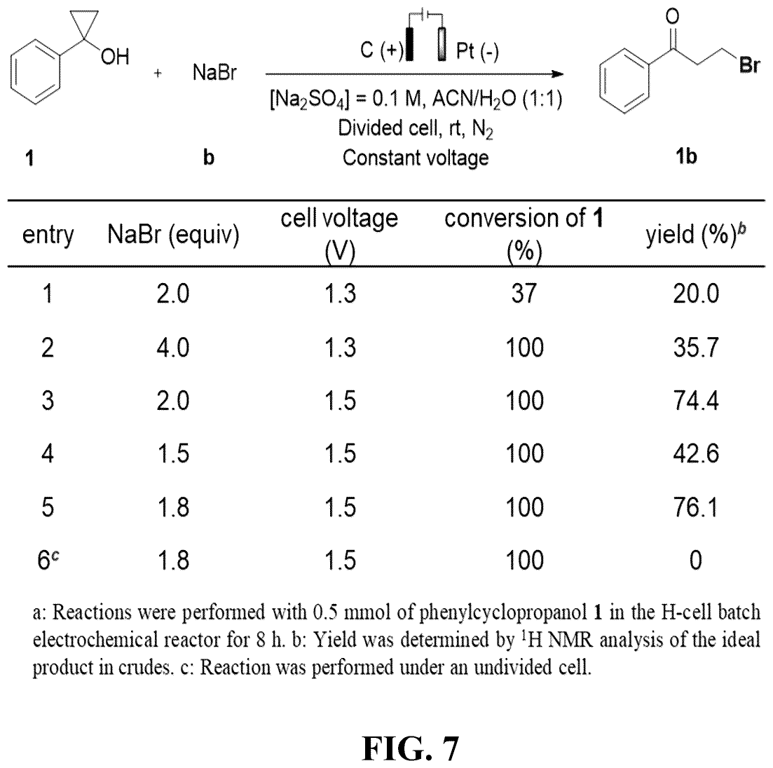

Once synthesized, the activity of various CPol derivatives was screened by conducting a variety of ring-opening reactions with NaBr and phenylcyclopropanol I-1 as model substrates in CH₃CN/H₂O (1:1) solvent and under different cell potentials using proton nuclear magnetic resonance (¹H NMR) analysis of substrate conversion (FIGS. 6-7). The screening results demonstrated that phenylcyclopropanol I-1 was completely consumed and conversed to the desired ketone product in the ¹H NMR yield of 76.1% when using 1.8 eq. of NaBr and constant voltage of 1.5 V (vs. SCE) in the electrolyte of 0.1 M Na₂SO₄ in a H-cell batch.

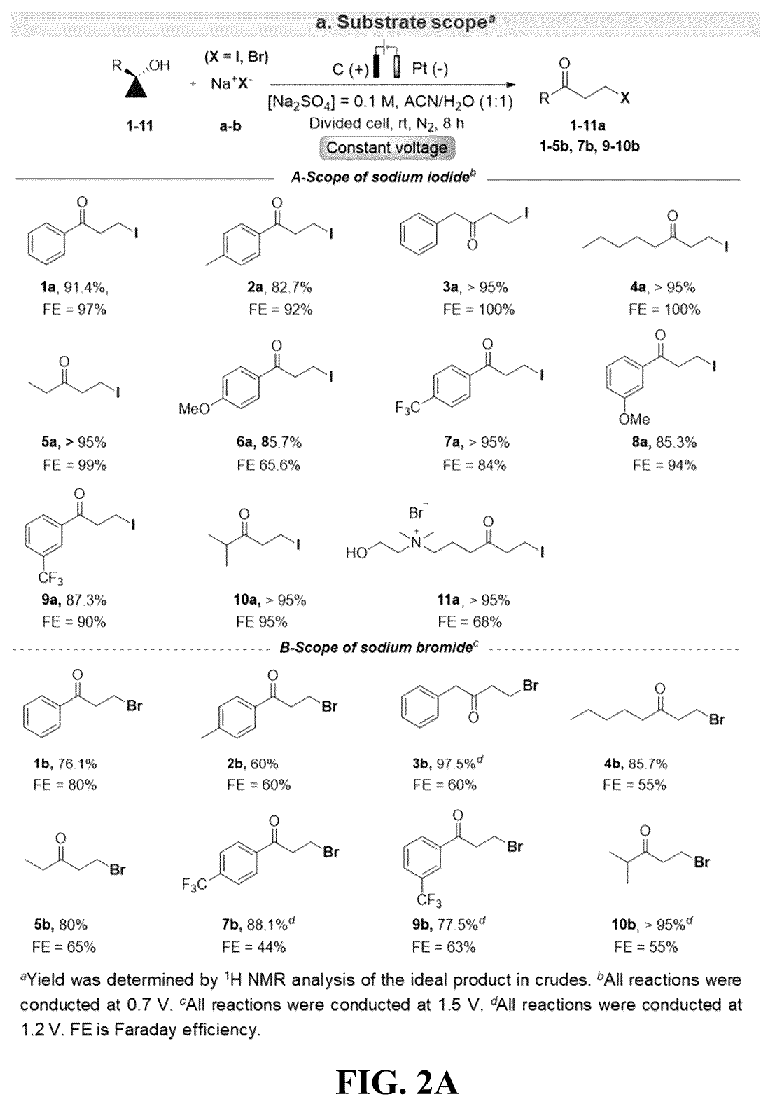

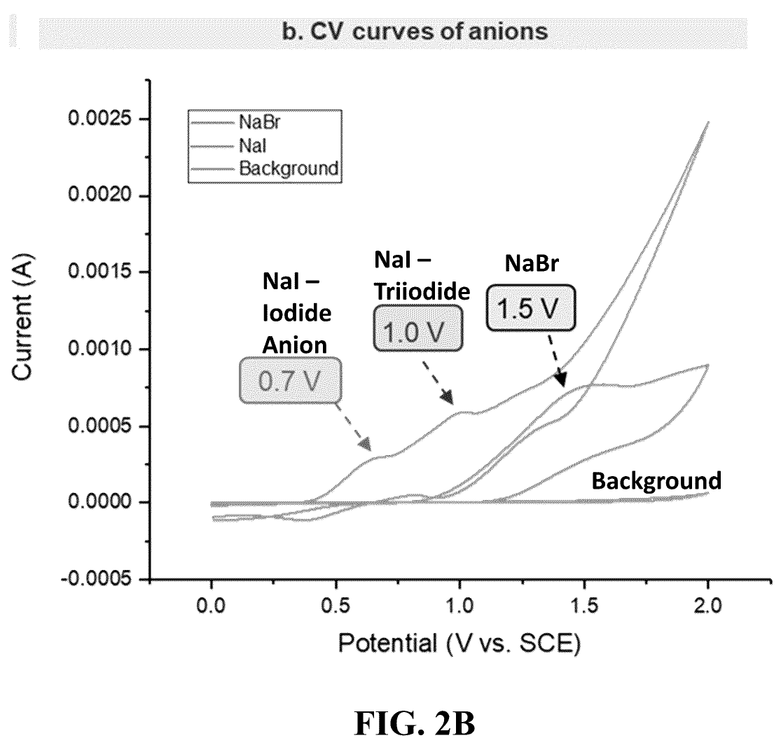

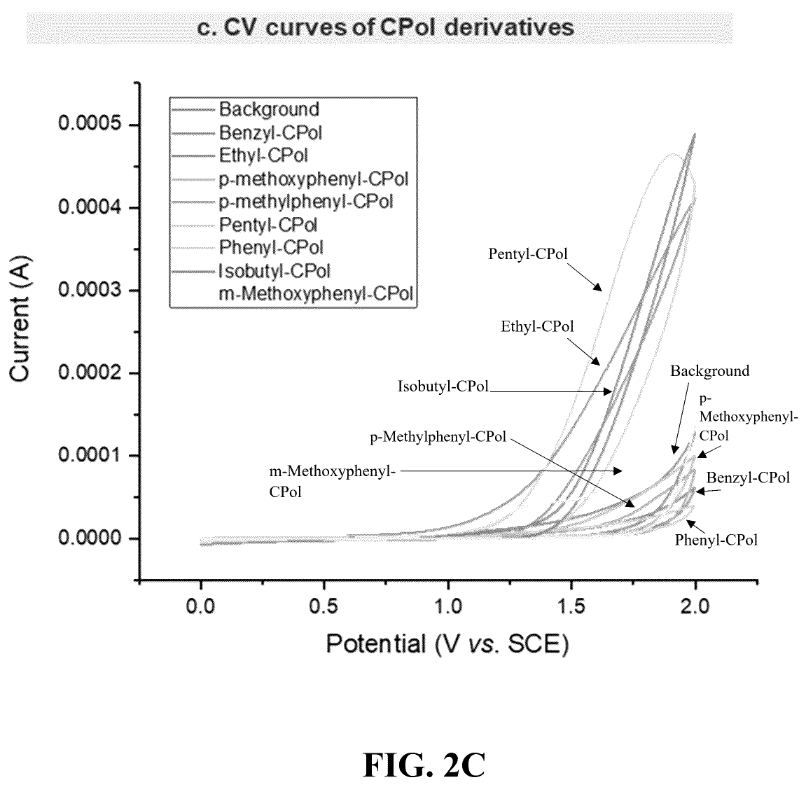

Next, the applicability of the ring-opening reaction while using different CPol derivatives and anions was evaluated. Based on the cyclic voltammetry (CV) curves (FIGS. 2B-2C), it was deduced that the energy required for the ring-opening of CPol derivatives is higher than that of NaI oxidation, and some of them are also higher than that of NaBr. Thus, ideally, the anions could be oxidized selectively without the oxidation of CPol derivatives. As shown in FIG. 2B, the oxidation of iodide anion (I⁻) and triiodide (I₃⁻) occurs at approximately 0.7 V versus SCE and 1 V versus SCE respectively, while the oxidation of bromine anion (Br⁻) takes place at around 1.5 V versus SCE. Further screening of CPol derivatives and different anions based on the CV curve was evaluated (FIG. 2A). These CPol derivatives were treated with NaI and NaBr respectively under optimized electrochemical conditions to afford β-substituted ketone, and the results revealed that CPol derivatives with different electronic and steric properties are well tolerated and generated halide-substituted ketone in ¹H NMR yields of 60-97.5% and Faraday efficiency of 44%-100%. Of note, the reduced ¹H NMR yield and Faraday efficiency may arise from the radical nature of the halogen likely consuming current in occasion non-productive pathways. Meaning that not every generated Br or I radical leads to the desired product formation. Moreover, the elevated oxidation potential of NaBr may lead to a competing reaction involving the ring-opening of CPol itself, which may be another reason for the lower ¹H NMR yield of the NaBr reaction.

Characterization data of the resulting electrophilic warheads are described below:

3-iodo-1-phenylpropan-1-one (I-1a). ¹H NMR (400 MHz, CDCl₃) δ 8.01-7.94 (m, 2H), 7.62 (m, 1H), 7.51 (t, J=7.5 Hz, 2H), 3.67 ((t, J=6.9 Hz, 2H), 3.50 (t, J=6.9 Hz, 2H). ¹³C NMR (101 MHz, CDCl₃) δ 197.7; 136.3; 136.7; 128.9; 128.2; 42.8; −3.9. HRMS (ESI) calculated for C₉H₉IO [M+H]⁺ 254.9698 found 259.9706.

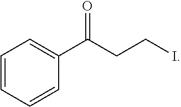 [SMILES: O=C(CCI)c1ccccc1]

3-iodo-1-(p-tolyl)propan-1-one (I-2a). ¹H NMR (400 MHz, CDCl₃) δ 7.85 (d, J=8.2 Hz, 2H), 7.27 (d, J=8.9 Hz, 3H), 3.61 (t, J=7.4 Hz, 2H), 3.51-3.43 (t, J=7.4 Hz, 2H), 2.42 (s, 3H). ¹³C NMR (101 MHz, CDCl₃) δ 197.4; 144.6; 133.8; 129.6; 128.3; 42.6; 21.8; −3.5. HRMS (ESI) calculated for C₁₀H₁₁IO [M+H]⁺ 273.9855 found 273.9849.

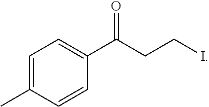

4-iodo-1-phenylbutan-2-one (I-3a). ¹H NMR (400 MHz, CDCl₃) δ 7.38-7.31 (m, 2H), 7.31-7.27 (m, 1H), 7.20 (d, J=7.0 Hz, 2H), 3.70 (s, 2H), 3.25 (t, J=7.0 Hz, 2H), 3.09 (t, J=7.0 Hz, 2H). ¹³C NMR (101 MHz, CDCl₃) δ 205.7; 133.5; 129.6; 129.0; 127.5; 50.3; 45.5; −4.4.

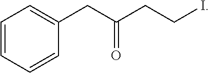 [SMILES: O=C(CCI)Cc1ccccc1]

1-iodooctan-3-one (I-4a). ¹H NMR (400 MHz, CDCl₃) δ 3.29 (t, J=7.0 Hz, 2H), 3.06 (t, J=7.0 Hz, 2H), 2.40 (t, J=7.4 Hz, 2H), 1.66-1.54 (m, 3H), 1.39-1.21 (m, 5H), 0.89 (t, J=7.0 Hz, 3H). ¹³C NMR (101 MHz, CDCl₃) δ 208.5; 46.3; 43.1; 31.5; 23.4; 22.6; 14.0; −4.1. HRMS (ESI) calculated for C₈H₁₅IO [M+H]⁺ 254.0168 found 254.0169.

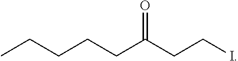

1-iodopentan-3-one (I-5a). ¹H NMR (400 MHz, CDCl₃) δ 3.30 (t, J=7.0 Hz, 2H), 3.07 (t, J=7.0 Hz, 2H), 2.44 (q, J=7.3 Hz, 2H), 1.08 (t, J=7.3 Hz, 3H). ¹³C NMR (101 MHz, CDCl₃) δ 208.8; 45.9; 36.3; 7.7; −4.1. HRMS (ESI) calculated for C₅H₉IO [M+H]⁺ 211.9698 found 211.9693.

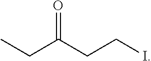

3-iodo-1-(4-methoxyphenyl)propan-1-one (I-6a). ¹H NMR (400 MHz, CDCl₃) δ 7.93 (d, J=8.9 Hz, 2H), 6.95 (d, J=8.9 Hz, 2H), 3.88 (s, 3H), 3.58 (t, J=7.5 Hz, 2H), 3.50-3.44 (t, J=7.5 Hz, 2H). ¹³C NMR (101 MHz, CDCl₃) δ 196.1; 163.9; 130.4; 129.3; 113.9; 55.5; 42.2; −3.4. HRMS (ESI) calculated for C₁₀H₁₁IO₂ [M+H]⁺ 289.9804 found 289.9811.

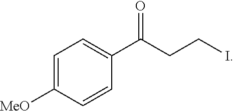 [SMILES: COc1ccc(C(=O)CCI)cc1]

3-iodo-1-(4-(trifluoromethyl)phenyl)propan-1-one (I-7a). ¹H NMR (400 MHz, CDCl₃) δ 8.05 (d, J=8.3 Hz, 2H), 7.75 (d, J=8.3 Hz, 2H), 3.66 (t, J=6.9 Hz, 2H), 3.48 (t, J=7.0 Hz, 2H). ¹³C NMR (101 MHz, CDCl₃) δ 196.8; 128.5; 126.01 (q, J=3.7 Hz); 43.0; −4.8. HRMS (ESI) calculated for C₁₀H₈F₃IO [M+H]⁺ 327.9572 found 327.9583.

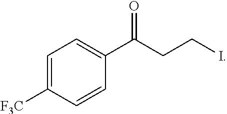 [SMILES: O=C(CCI)c1ccc(C(F)(F)F)cc1]

3-iodo-1-(3-methoxyphenyl)propan-1-one (I-8a). ¹H NMR (400 MHz, CDCl₃) δ 7.50 (dd, J=12.6, 5.0 Hz, 2H), 7.38 (t, J=7.9 Hz, 1H), 7.16-7.10 (m, 1H), 3.87 (s, 3H), 3.62 (t, J=6.9 Hz, 2H), 3.47 (t, J=7.1 Hz, 2H).

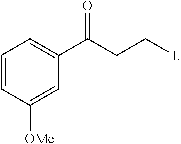 [SMILES: COc1cccc(C(=O)CCI)c1]

3-iodo-1-(3-(trifluoromethyl)phenyl)propan-1-one (I-9a). ¹H NMR (400 MHz, CDCl₃) δ 8.20 (s, 1H), 8.13 (d, J=7.8 Hz, 1H), 7.85 (d, J=7.7 Hz, 1H), 7.64 (t, J=7.8 Hz, 1H), 3.66 (t, J=6.9 Hz, 2H), 3.48 (t, J=7.0 Hz, 2H). ¹³C NMR (101 MHz, CDCl₃) δ 196.4; 136.8; 131.3; 130.1 (d, J=3.6 Hz); 129.6; 125.0 (d, J=3.6 Hz); 42.8; −4.7.

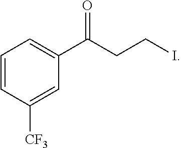 [SMILES: O=C(CCI)c1cccc(C(F)(F)F)c1]

1-iodo-4-methylpentan-3-one (I-10a). ¹H NMR (400 MHz, CDCl₃) δ 3.30 (t, J=6.9 Hz, 2H), 3.12 (t, J=7.0 Hz, 2H), 2.58 (m, 1H), 1.12 (d, J=6.9 Hz, 6H). ¹³C NMR (101 MHz, CDCl₃) δ 211.9; 44.1; 41.0; 18.0; 1.2; −3.8.

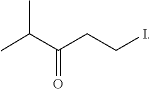

N-(2-hydroxyethyl)-6-iodo-N,N-dimethyl-4-oxohexan-1-aminium bromide (I-11a). ¹H NMR (400 MHz, CD₃CN) δ 3.96 (m, 2H), 3.41-3.37 (m, 2H), 3.33 (t, J=6.7 Hz, 2H), 3.31-3.25 (m, 2H), 3.14 (t, J=6.7 Hz, 2H), 3.08 (s, 6H), 2.55 (t, J=6.8 Hz, 2H), 2.03-1.98 (m, 2H). ¹³C NMR (101 MHz, CD₃CN) δ 207.6; 118.3; 66.3; 65.3; 56.6; 52.6-52.5 (m); 46.5; 39.1; 17.4; −2.9. HRMS (ESI) calculated for C₁₀H₂₁INO₂ [M+H]⁺ 314.0612 found 314.0611.

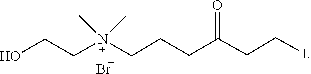

3-bromo-1-phenylpropan-1-one (I-1b). ¹H NMR (400 MHz, CDCl₃) δ 7.99-7.93 (m, 2H), 7.60 (t, J=7.4 Hz, 1H), 7.49 (t, J=7.7 Hz, 2H), 3.75 (t, J=6.9 Hz, 2H), 3.58 (t, J=6.9 Hz, 2H). ¹³C NMR (101 MHz, CDCl₃) δ 197.1; 136.4; 133.7; 128.9; 128.2; 41.7; 25.9. HRMS (ESI) calculated for C₉H₉BrO [M+H]⁺ 211.9837 found 211.9839.

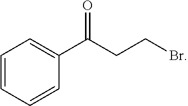 [SMILES: O=C(CCBr)c1ccccc1]

3-bromo-1-(p-tolyl)propan-1-one (I-2b). ¹H NMR (400 MHz, CDCl₃) δ 7.86 (d, J=8.2 Hz, 2H), 7.30-7.25 (m, 3H), 3.74 (t, J=6.9 Hz, 2H), 3.55 (t, J=7.0 Hz, 2H), 2.42 (s, 4H). ¹³C NMR (101 MHz, CDCl₃) δ 196.8; 144.6; 134.0; 129.6; 128.3; 41.6; 26.1; 21.8. HRMS (ESI) calculated for C₁₀H₁₁BrO [M+H]⁺ 225.9993 found 225.9996.

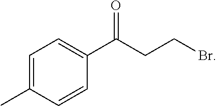

4-bromo-1-phenylbutan-2-one (I-3b). ¹H NMR (400 MHz, CDCl₃) δ 7.99-7.93 (m, 2H), 7.60 (t, J=7.4 Hz, 1H), 7.49 (t, J=7.7 Hz, 2H), 3.75 (t, J=6.9 Hz, 2H), 3.58 (t, J=6.9 Hz, 2H). ¹³C NMR (75 MHz, CDCl₃) δ 205.3; 133.5; 129.6; 129.0; 127.5; 50.6; 44.5; 25.4.

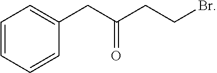 [SMILES: O=C(CCBr)Cc1ccccc1]

1-bromooctan-3-one (I-4b). ¹H NMR (400 MHz, CDCl₃) δ 3.56 (t, J=6.8 Hz, 2H), 3.00 (t, J=6.8 Hz, 2H), 2.43 (t, J=7.4 Hz, 2H), 1.64-1.55 (m, 2H), 1.37-1.23 (m, 5H), 0.93-0.86 (m, 4H). ¹³C NMR (101 MHz, CDCl₃) δ 208.0; 45.3; 43.4; 31.4; 25.6; 23.4; 22.6; 14.0.

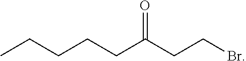

1-bromopentan-3-one (I-5b). ¹H NMR (400 MHz, CDCl₃) δ 3.56 (t, J=6.8 Hz, 2H), 3.00 (t, J=6.8 Hz, 2H), 2.46 (q, J=7.3 Hz, 2H), 1.08 (t, J=7.3 Hz, 3H). ¹³C NMR (101 MHz, CDCl₃) δ 208.3; 44.9; 36.6; 25.6; 7.6.

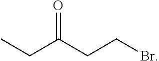

3-bromo-1-(4-(trifluoromethyl)phenyl)propan-1-one (I-7b). ¹H NMR (400 MHz, CDCl₃) δ 8.06 (d, J=8.1 Hz, 2H), 7.75 (d, J=8.2 Hz, 2H), 3.75 (t, J=6.9 Hz, 2H), 3.60 (t, J=6.9 Hz, 2H).

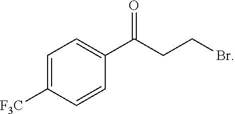 [SMILES: O=C(CCBr)c1ccc(C(F)(F)F)cc1]

3-bromo-1-(3-(trifluoromethyl)phenyl)propan-1-one (I-9b). ¹H NMR (400 MHz, CDCl₃) δ 8.20 (s, 1H), 8.14 (d, J=7.8 Hz, 1H), 7.85 (d, J=7.8 Hz, 1H), 7.64 (t, J=7.8 Hz, 1H), 3.75 (t, J=6.9 Hz, 2H), 3.60 (t, J=6.9 Hz, 2H).

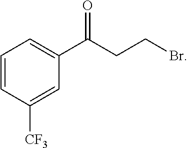 [SMILES: O=C(CCBr)c1cccc(C(F)(F)F)c1]

1-bromo-4-methylpentan-3-one (I-10b). ¹H NMR (400 MHz, CDCl₃) δ 3.56 (t, J=6.8 Hz, 2H), 3.05 (t, J=6.8 Hz, 2H), 2.59 (m, 1H), 1.11 (d, J=6.9 Hz, 6H). ¹³C NMR (101 MHz, CDCl₃) δ 211.4; 43.0; 41.2; 25.8; 18.0.

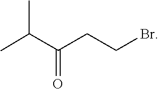

### Example 3

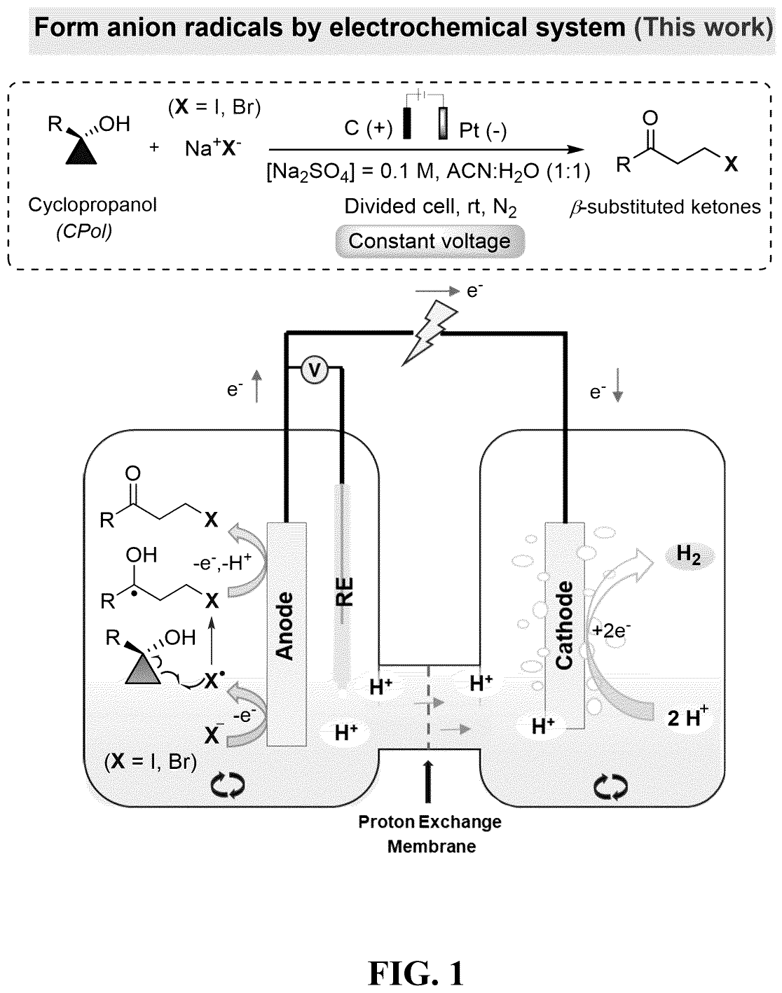

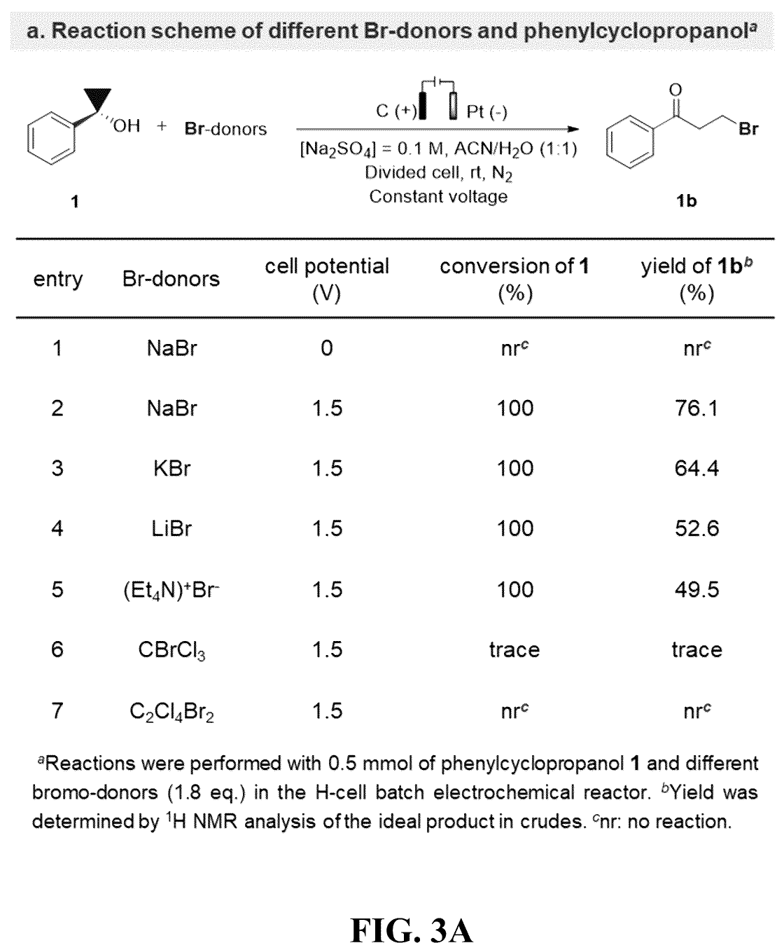

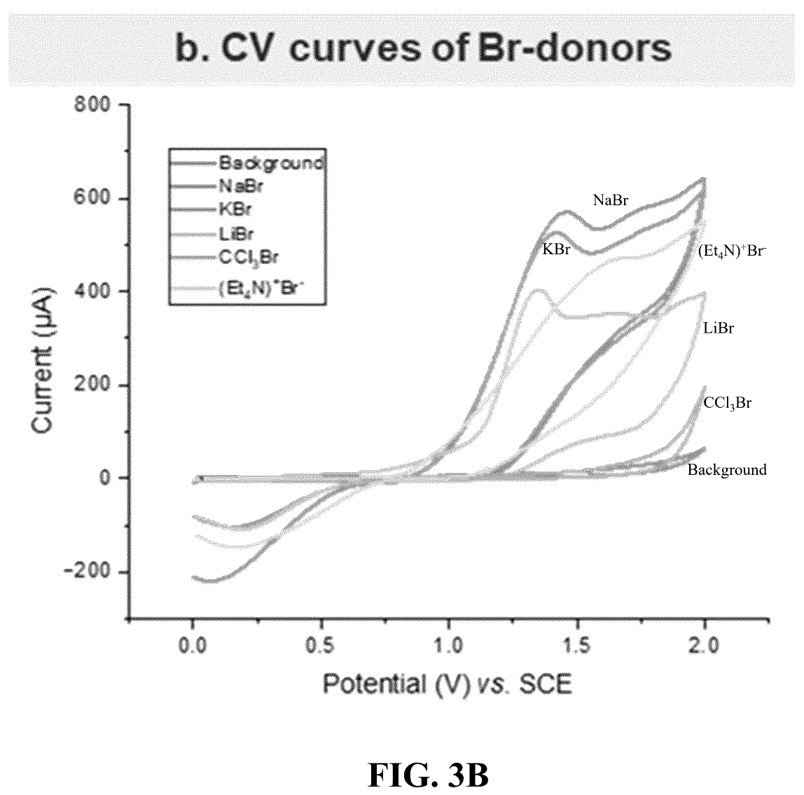

To obtain more insights into the ring opening reaction mechanism of CPol under electrochemical conditions, a series of mechanistic studies were conducted (FIG. 3A). The oxidation potential value of the individual Bromo (Br)-donor will be collected via CV measurements (FIG. 3B). And the reactions between Br-donors and phenylcyclopropanol were conducted under optimized electrochemical conditions. The outcomes revealed that phenylcyclopropanol and bromide (Br⁻) do not yield the desired product without electrochemical initiation (FIG. 3A). Likewise, the reaction between bromo cation (Br⁺) and phenylcyclopropanol did not generate the ketone product under optimized electrochemical conditions (FIG. 3A). Nonetheless, the bromo radical (Br^{·}) derived from various bromide salts efficiently promotes the ring opening of phenylcyclopropanol under the same conditions (FIG. 3A). This leads us to propose that anion radicals play a vital role in the ring-opening reaction of CPol, and a plausible mechanism is proposed, as illustrated in FIG. 1. Once the formation of anion radicals, the CPol ring was opened via the β-scission process generating a tertiary carbon radical. The radical species was subsequently oxidized on the electrode, ultimately yielding the desired ketone product.

### Example 4

### Electrochemically Promoted Bioconjugation

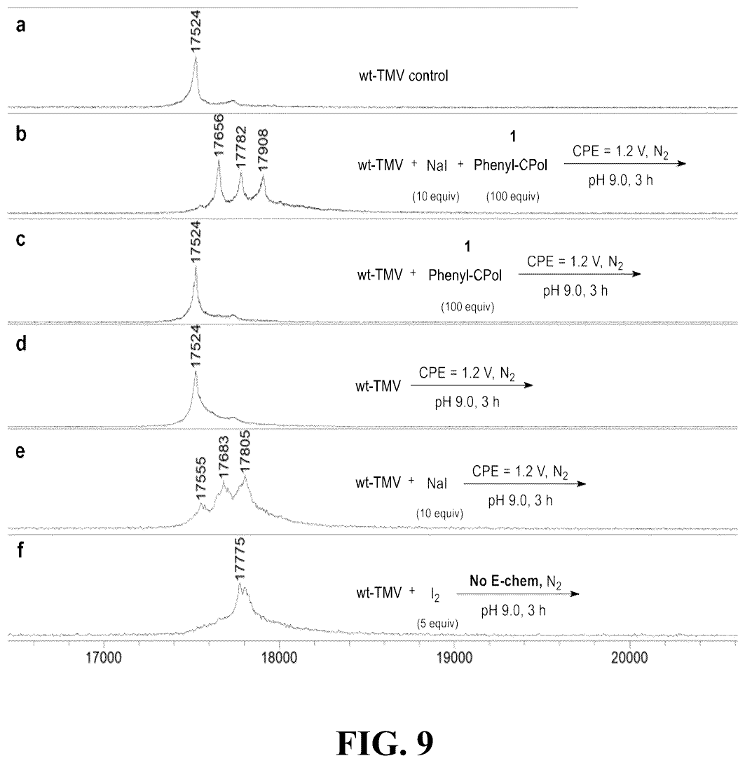

In the subsequent investigation, to prove the availability and selectivity of CPol warhead in bioconjugation, plant virus tobacco mosaic virus (TMV) was chosen as a multivalent protein scaffold to demonstrate the bioconjugation strategy due to its well-defined structure and the ability to be easily manipulated.²⁴ At the outset, we treated wide-type (wt) TMV with CPol derivatives (10 eq.) using various conditions, aiming to investigate the potential of CPol derivatives for bioconjugation (FIG. 9). Fortunately, the phenylcyclopropanol I-1 modified TMV peak ([M+132]⁺) was found in the MALDI-TOF MS spectrum upon the introduction of NaI at a potential of 1.2 V (FIG. 9). To establish the significance of 1 in this bioconjugation process, we subjected the reaction between wt-TMV and NaI to a potential of 1.2 V without the addition of 1. As a result, we observed the formation of iodide-substituted TMV instead of 1-conjugated-TMV (FIG. 9). Similarly, to demonstrate the significance of the NaI redox catalyst, the wt-TMV and 1 were combined under identical electrochemical conditions. Unsurprisingly, no reaction occurred (FIG. 9). Subsequently, to eliminate the influence of the iodine catalyst during the bioconjugation process, the wt-TMV was treated solely with iodine without electrochemical stimulation. The results revealed the exclusive formation of diiodide-substituted TMV without the formation of 1-conjugated TMV (FIG. 9). Furthermore, the stability of wt-TMV under the same electrochemical conditions was also confirmed (FIG. 9).

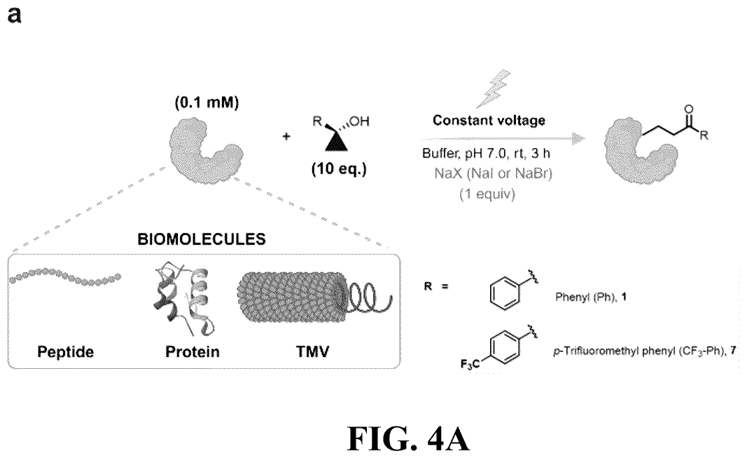

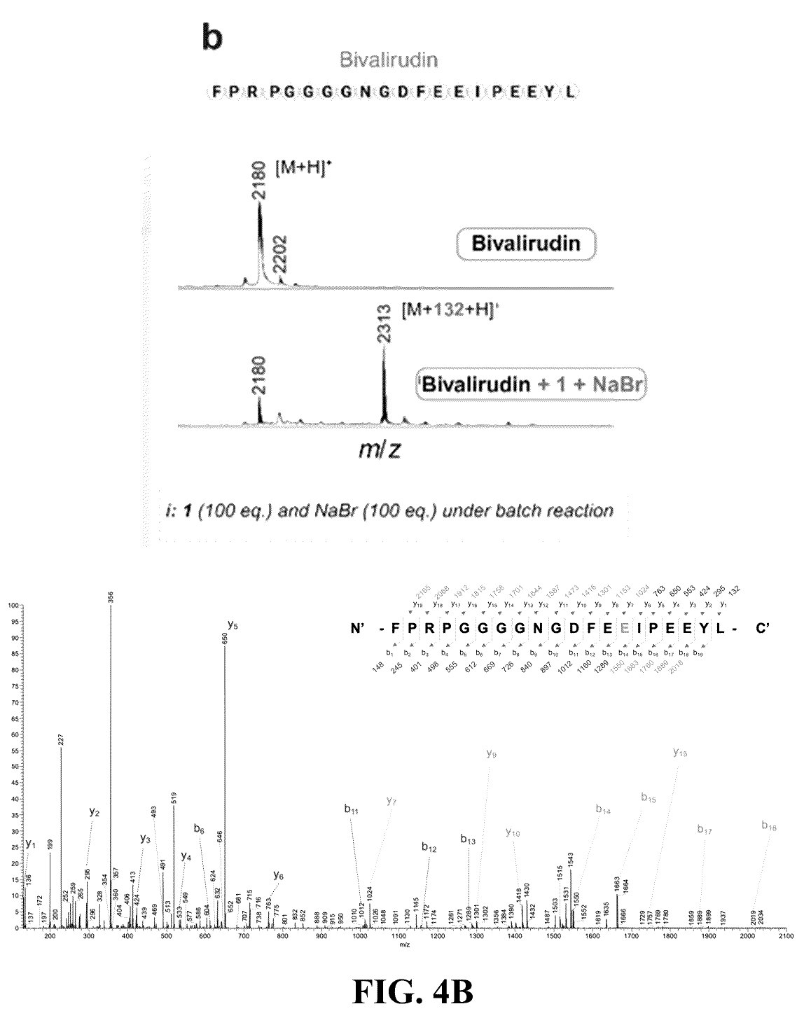

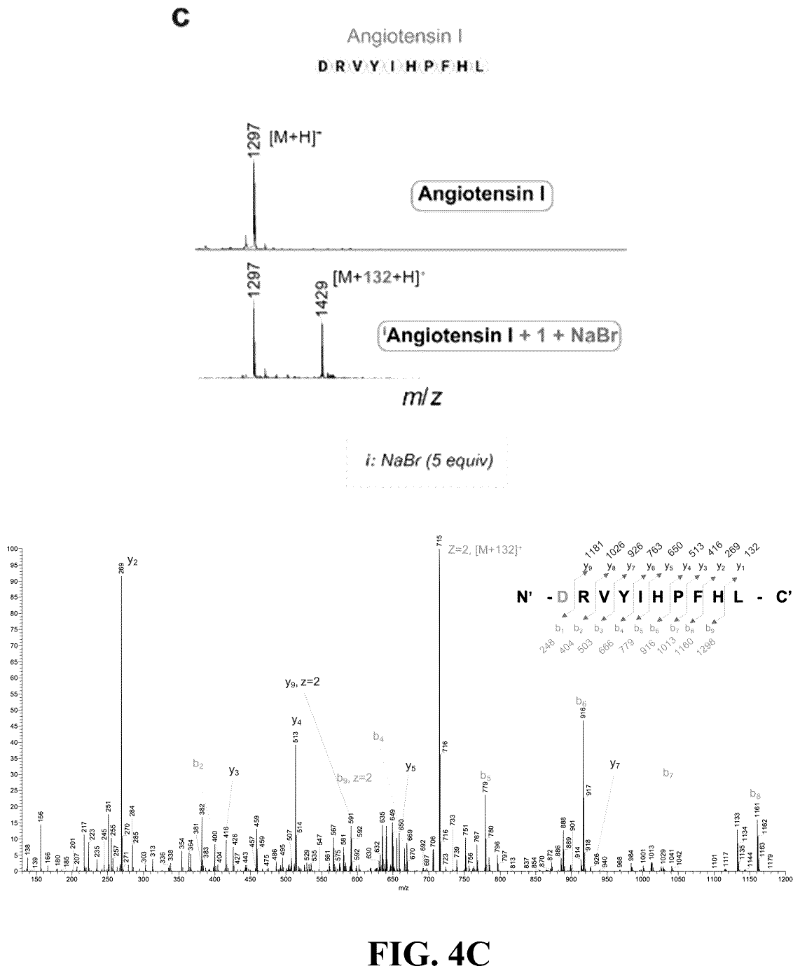

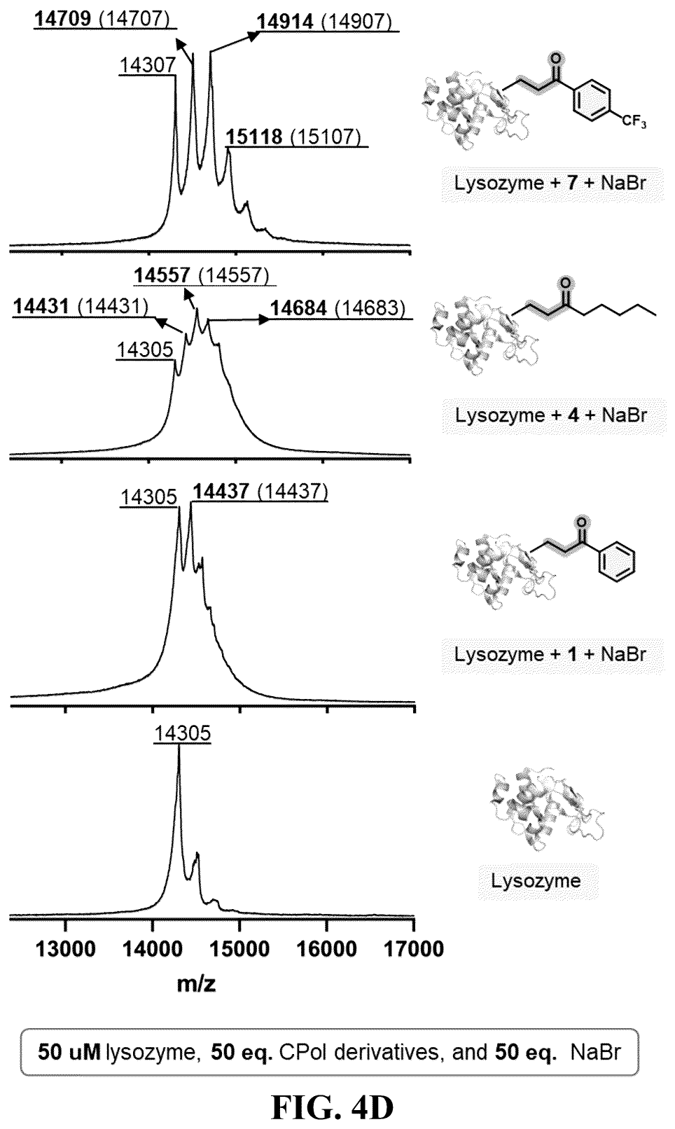

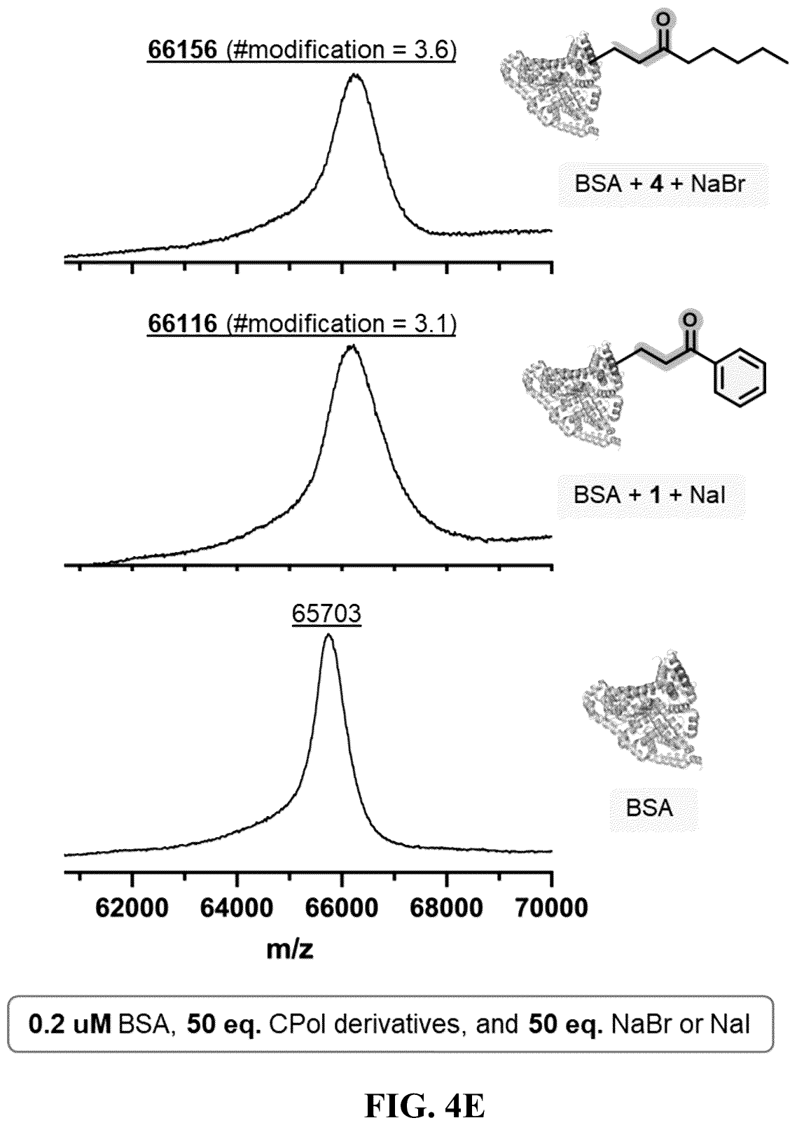

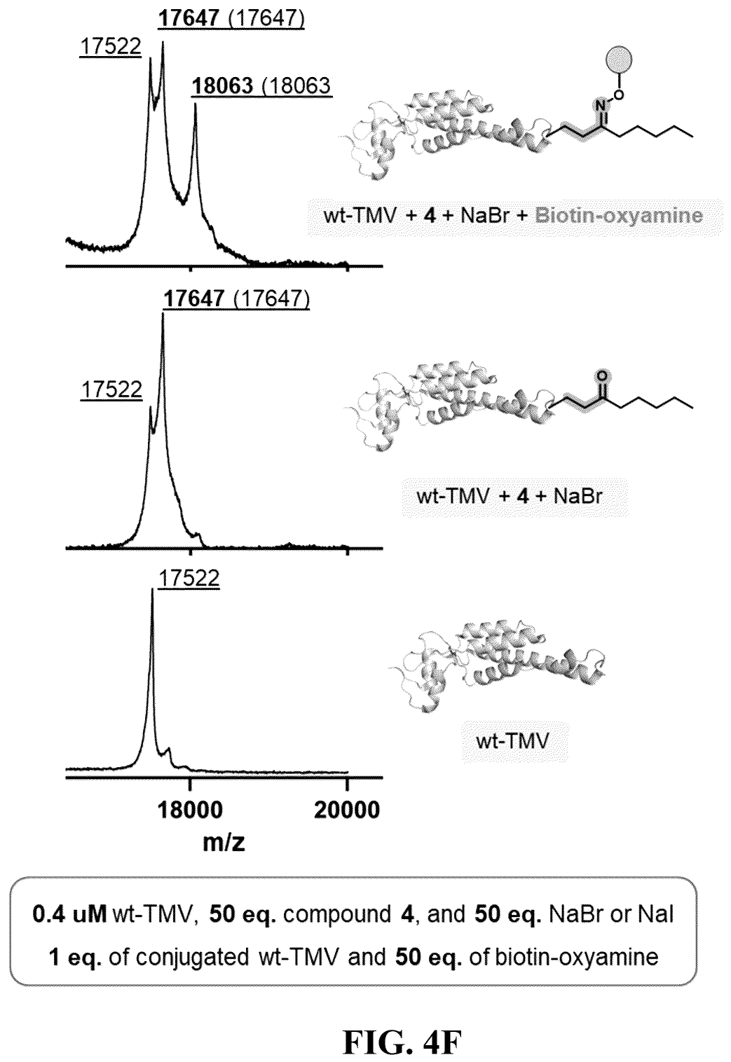

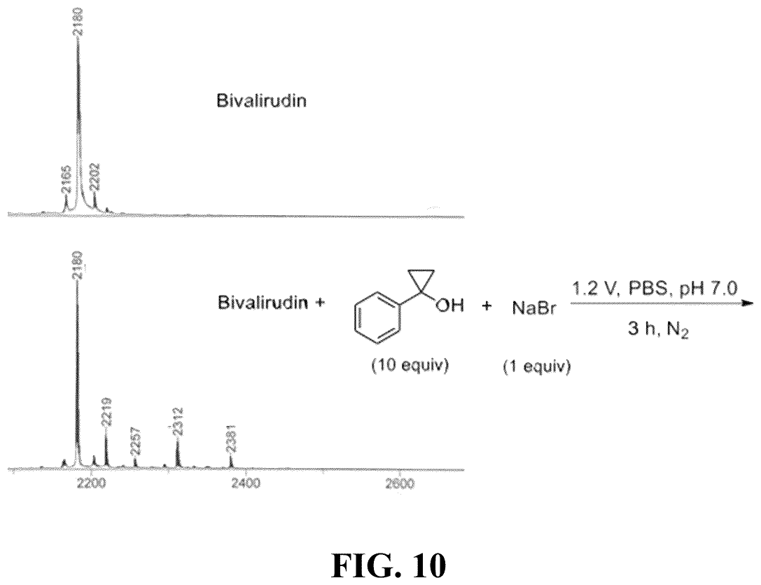

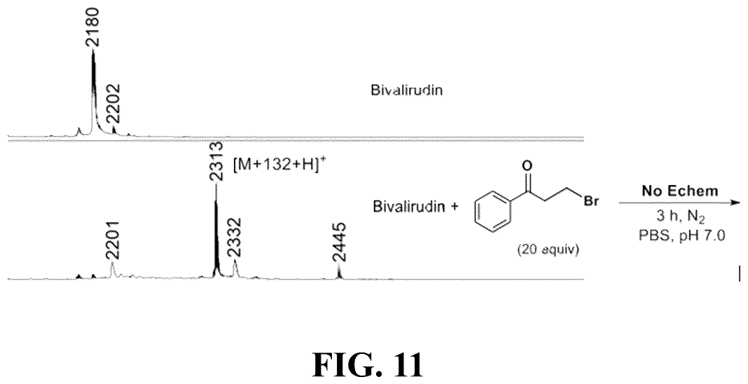

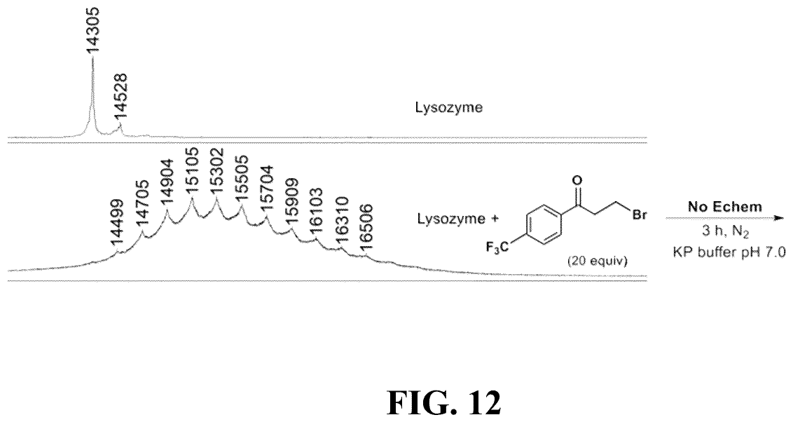

To further access the compatibility of biomolecules and various CPol derivatives within the electrochemical bioconjugation process, a range of biomolecules including peptides, proteins, and TMV, along with different CPol derivatives, were subjected to the NaI or NaBr redox catalyst and a low cell potential of 0.8 V or 1.2 V individually (FIG. 4A). Electrochemical bioconjugation was employed for labeling angiotensin I, which is a peptide hormone responsible for vasoconstriction and evaluated in blood pressure. The results demonstrated the successful labeling of angiotensin I using Formula I-1 and NaBr under constant voltage, and the modified amino acid site was identified as glutamic acid (Glu, E) on angiotensin I using LC-MS (FIG. 4C). However, when modifying angiotensin I with NaI at a voltage of 0.8V, certain side reactions may occur. Similarly, the majority of bivalirudin peptides can be effectively modified by employing NaBr and maintaining a constant voltage of 1.2 V within a batch reaction environment, rather than using a one-pot reaction condition (FIG. 4B). The modified amino acid site was identified as an aspartic acid (Asp, D) residue on bivalirudin using LC-MS. The conversion yield of bivalirudin is notably reduced in a one-pot reaction condition, primarily due to the formation of side products in an aqueous solution (FIG. 10), which makes us reconsider the bioconjugation mechanism. Thus, we conducted a preliminary reaction involving bivalirudin or lysozyme, compound 1, and the resulting Br-substituted ketone without the application of electrochemical conditions (FIGS. 11-12). As a result, we found that all bivalirudin or lysozyme underwent modification, indicating that the in-situ formation of β-substituted ketone under electrochemical conditions can promote this bioconjugation reaction.

Subsequently, the electrochemical bioconjugation approach was evaluated on lysozyme, a 14 kDa cationic protein. It was observed that I-1, I-4, or I-7 were efficiently conjugated to lysozyme utilizing various electrochemical conditions (FIG. 4D). Additionally, the electrochemical bioconjugation on bovine serum albumin (BSA) was also evaluated. It was shown that I-1 and I-4 were efficiently conjugated into BSA, a 66 kDa protein (FIG. 4E). Furthermore, the versatility of TMV conjugation was explored, leading to the discovery that compound I-4 was successfully modified to wt-TMV (m/z=17647 in FIG. 4F). Besides, the conjugated biomolecule was conjugated with biotin-oxyamine under weak acidic conditions (m/z=18063 in FIG. 4F), which can be used in further cell labeling or protein proteomic analysis. In summary, the CPol bioconjugation warhead exhibits specific amino acid selectivity and can be conjugated with various biomolecules under electrochemical bioconjugation approach has the potential to be a valuable bioorthogonal tool for biological studies.

### Example 5

### Electrochemically Assisted Cell Labeling

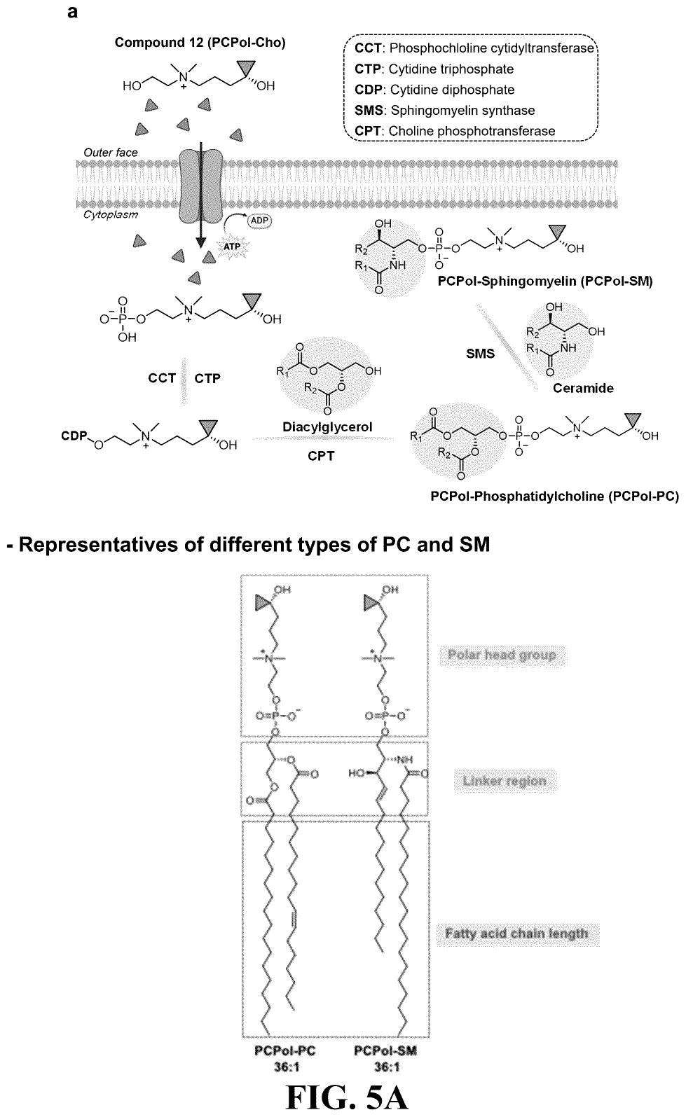

To explore whether the CPol building block can be a bioorthogonal tag in cell labeling, first the CPol-choline analog was synthesized which bears a terminal CPol moiety, followed by its incorporation into mammalian cells via the biosynthetic process (FIG. 5A).

### Synthesis of CPol-Choline Analog:

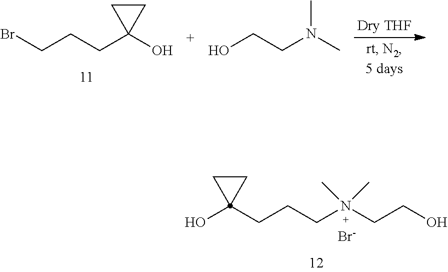

3-bromopropyl cyclopropanol (11, 2.79 mmol, 0.5 g) and N,N-dimethylethanolamine (1.611 mmol, 143.6 mg) were added into a vial under N₂ protection. Dry THF (6 mL) was added to this vial. The reaction was stirred at room temperature for 5 days. After 5 days, the THF in the vial was removed through rotavapor at room temperature to get colorless oil. The resultant oil was washed with diethyl ether at least 20 times until all starting materials were washed away (2 mL per time). Subsequently, the solvents in the washed oil were removed through rotavapor to afford CPol-choline analog 12, Formula I-12. The CPol-choline analog may have the following Formula (I-12):

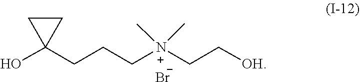

CPol-choline analog (I-12). Colorless oil was obtained in 84.3% isolated yield. ¹H NMR (400 MHz, MeOD) δ 4.01 (m, 2H), 3.53-3.47 (m, 4H), 3.19 (s, 6H), 2.10-1.98 (m, 2H), 1.60 (t, J=7.4 Hz, 2H), 0.71 (m, 2H), 0.49 (m, 2H). ¹³C NMR (101 MHz, MeOD) δ 66.9-66.4 (m); 56.9; 54.8; 52.5-52.1 (m); 35.9; 20.7; 13.8. HRMS (ESI) calculated for C₁₀H₂₂NO₂ [M+H]⁺ 188.1651 found 188.1645.

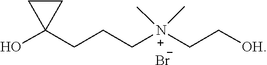

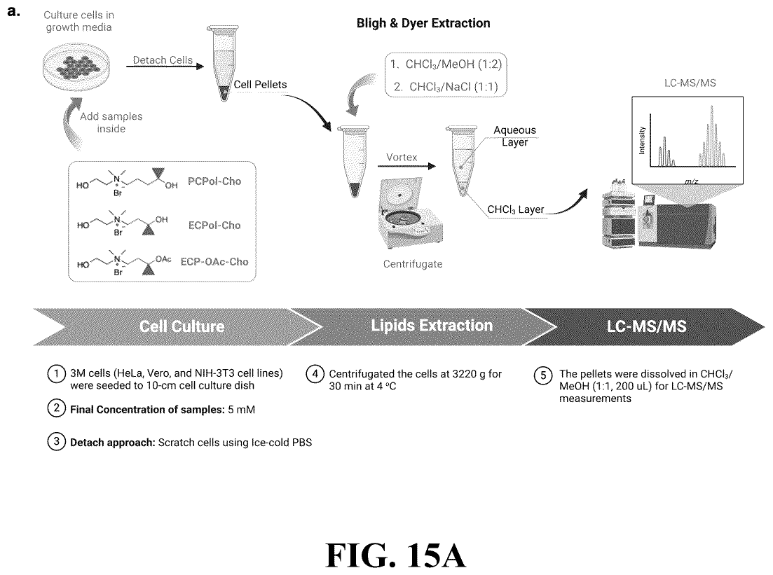

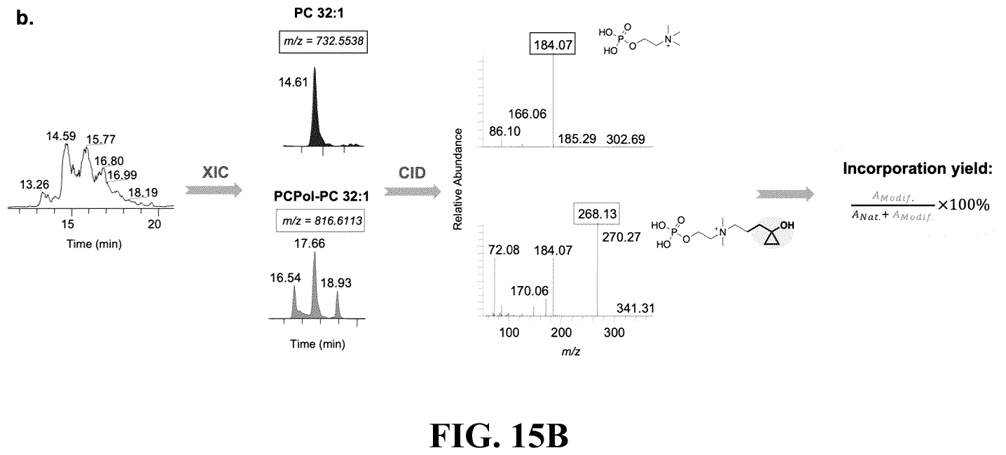

Choline stands as the predominant head group in phospholipids which are the principal components of all cellular membranes. Due to its comparable structure with natural choline, CPol-choline analog will likewise be used by cells in place of choline via biological metabolism, efficiently serving as a phospholipid precursor. (FIG. 5A). The metabolic incorporation efficiency of CPol-choline was evaluated through lipidomic experiments (FIG. 15A) and LC-MS (FIG. 15B). The results demonstrated that CPol-choline was successfully incorporated into mammalian cells, such as Hela, Vero, and NIH-3T3 cells (FIG. 5B), facilitating subsequent labeling experiments.

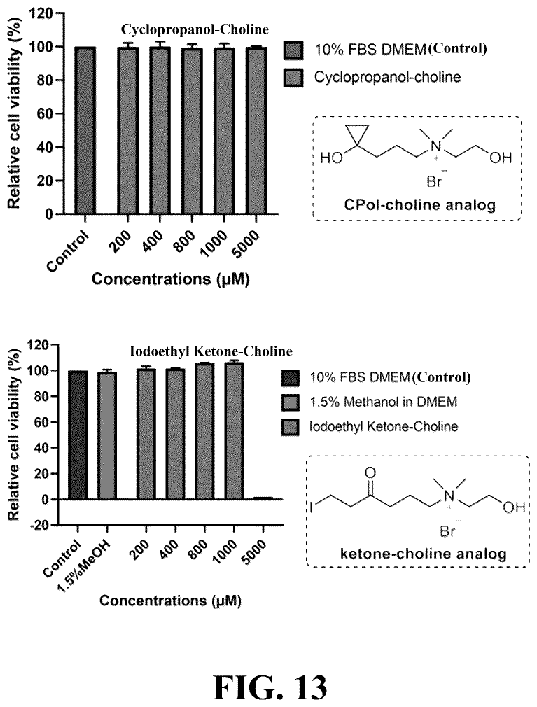

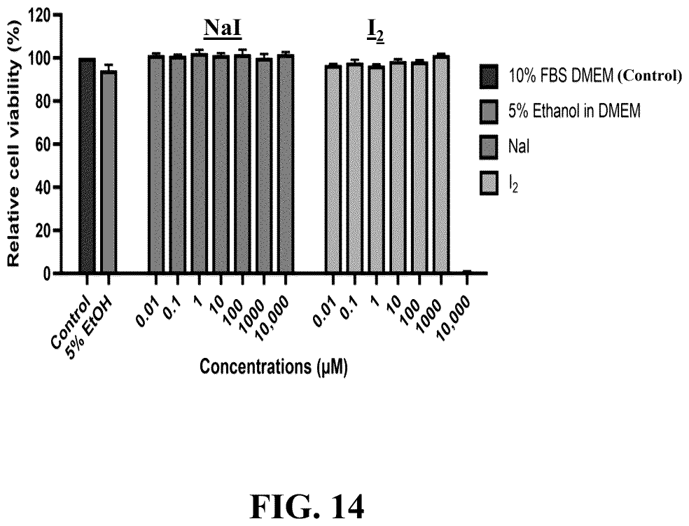

The generated phospholipid containing CPol moiety was stimulated by a mild electrochemical system to produce ketone moieties. The formed ketone was labeled with probes containing hydrazide or aminooxy groups for identification of cellular behaviors. Initially, the cytotoxicity of choline analogs, NaI, and iodine on Vero cells was evaluated using the CellTiter-Blue Cell Viability Assay (FIGS. 13-14). The outcomes indicated that no cytotoxicity was observed even in the highest concentration of CPol-choline analog at 5 mM, and NaI also can be safely co-cultured with Vero cells. Following the metabolic incorporation of the CPol-choline analog into Vero cells, the NaI solution was added to the resulting conjugated Vero cells and subjected to a constant voltage of 0.7 V. This process promoted the ring-opening of CPol and the formation of ketone functional groups on the cell membrane. Subsequently, the produced ketone entities were allowed to react with a hydrazide-containing fluorescence dye, fluorescein thiosemicarbazide (FTSCA), yielding a fluorescence turn-on response.

### Synthesis of Fluorescein Thiosemicarbazide (FTSCA):

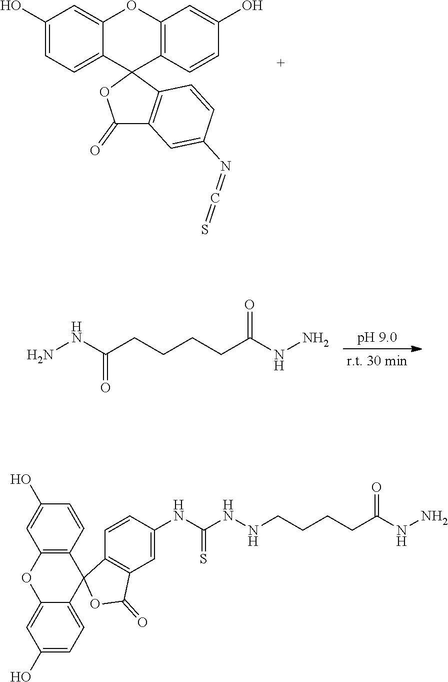 [SMILES: O=C1OC2(c3ccc(O)cc3Oc3cc(O)ccc32)c2ccc(N=C=S)cc21 ; NNC(=O)CCCCC(=O)NN ; NNC(=O)CCCCNNC(=S)Nc1ccc2c(c1)C(=O)OC21c2ccc(O)cc2Oc2cc(O)ccc21]

To 10 mL of 2.5% (w/v) adipic acid dihydrazide solution, which was adjusted to pH 9.0, 50 mL of 0.2% (w/v) fluorescein isothiocyanate in 50 mM bicarbonate-carbonate buffer (pH 9.0) was added dropwise with stirring. The mixture was kept at room temperature for 30 min; then 1 M glycine (pH 9.0, 6 mL) was added, and the mixture was kept at room temperature for another 30 min. After incubation, the mixture was adjusted to pH 3.4 using 20% HCl (v/v). The precipitate was collected by filtration and washed thoroughly with H₂O. Finally, the precipitate was dried in a vacuum and stored at −20° C. without light. The mixture was analyzed by mass spectrometry.

The intensity of this fluorescence was quantified using a plate reader and visualized using fluorescence microscopy (FIG. 6A). The intracellular fluorescence measurement results showed that the “CPol+NaI+Echem” group exhibited the strongest fluorescent intensity after labeling with FTSCA, indicating the successful incorporation of the CPol-choline analog into Vero cells (FIG. 6B). These conclusions were consistently supported by the results obtained from fluorescent imaging analyses (FIG. 6C). Therefore, the electroactive CPol warhead provides a valuable tool for real-time imaging or various other bioconjugation applications.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A method of producing a chemoselectively modified biomolecule, the method comprising:
providing a biomolecule comprising a bioconjugation warhead at a specific site of the biomolecule, wherein the bioconjugation warhead comprises a cyclopropanol derivative thereof and a halogen donor compound;
selectively oxidizing the halogen donor compound of the bioconjugation warhead at an electrochemically efficient voltage to generate a halogen radical;
contacting the halogen radical with the bioconjugation warhead at a constant voltage to form an electrophilic warhead; and
reacting the electrophilic warhead with a coupling partner comprising a nucleophilic group of the biomolecule to conjugate the electrophilic warhead to the biomolecule.

2. The method of claim 1, wherein the cyclopropanol derivative comprises Formula (I): 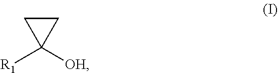
wherein R₁ may be selected from a group consisting of hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, and a combination thereof.

3. The method of claim 2, wherein the cyclopropanol derivative is selected from a group consisting of phenylcyclopropanol, 1-(p-tolyl)cyclopropan-1-ol, benzylcylopropanol, pentylcyclopropanol, 1-(4-methoxyphenyl)cyclopropan-1-ol, 1-(4-(trifluoromethyl)phenyl)cyclopropan-1-ol, 1-(3-methoxyphenyl)cyclopropan-1-ol, 1-(3-(trifluoromethyl)phenyl)cyclopropan-1-ol, 1-isopropylcyclopropan-1-ol, and 3-bromopropyl cyclopropanol.

4. The method of claim 1, wherein the halogen donor compound comprises a metal.

5. The method of claim 4, wherein the metal comprises sodium, lithium, potassium, rubidium, aluminum, ammonium, cesium, or francium.

6. The method of claim 4, wherein the halogen donor compound comprises sodium iodide, sodium bromide, lithium bromide, lithium iodide, potassium bromide, potassium iodine, tetraethylammonium bromide, tetraethylammonium chloride, or a combination thereof.

7. The method of claim 1, wherein the electrochemically efficient voltage is from about 0.1 V to about 2.0 V.

8. The method of claim 1, wherein an oxidation potential of the halogen donor compound is at least about 10% lower than an oxidation potential of the cyclopropanol derivative.

9. The method of claim 1, wherein selectively oxidizing the halogen donor compound of the bioconjugation warhead occurs in an electrochemical cell.

10. The method of claim 9, wherein the electrochemical cell is a divided cell.

11. A conjugated biomolecule, comprising: an electrophilic warhead conjugated to a specific site of a biomolecule, wherein the electrophilic warhead comprises the following formula: 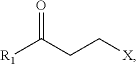
wherein R₁ may be selected from a group consisting of hydroxyl, amino, alkoxy, carboxy, benzyl, phenyl, nitro, thiol, thioalkoxy, halogen, alkyl, aryl, alkenyl, alkynyl, halogen, C₁₋₆alkyl, phenyl, perdeuterated phenyl, benzyl, cyclopropyl, cyclobutyl, pentyl, cyclopentyl, cyclohexyl, cyclopropylmethyl, cyclobutylmethyl, cyclopentylmethyl, cyclohexylmethyl, 2-pyridyl, 3-pyridyl, 4-pyridyl, 2-thiazolyl, 4-thiazolyl, 5-thiazolyl, 3-pyrazolyl, 4-pyrazolyl, 2-pyrimidinyl, 4-pyrimidinyl, 5-pyrimidinyl, 2-oxazolyl, 5-oxazolyl, 4-oxazolyl, 2-thiophenyl, 3-thiophenyl, 1-piperidinyl, 4-piperidinyl or 4-morpholinyl, 4-morpholinylcarbonyl, cyclopropylcarbonyl, 1-piperazinyl, 4-methyl-1-piperazinyl, 1-pyrrolidinyl, 1-piperazinylcarbonyl, 1-piperidinylcarbonyl, 1-pyrrolidinylcarbonyl, dimethylamino, 2-(4-morpholinyl)ethoxy, 1-(4-methoxy)phenyl, 3-methoxypropoxy, dimethylcarbamoyl, acetamido, propanoyl, 4-thiomorpholino, 4-thiomorpholino-S,S-oxide, 1-pyrrolidinyl, methylsulfonylamino, methylsulfonyl, propanoylamino, 1-cyclopentenyl, 1-cyclohexenyl, 1,2,3,6-tetrahydropyridin-4-yl, 1,2,3,6-tetrahydropyridin-5-yl, 2,5-dihydro-1H-pyrrol-3-yl, 2,5-dihydro-pyrrol-1-yl, 2-norbornyl, toyl, trifluoromethyl, difluoromethyl, fluoromethyl, 2,2,2-trifluoroethyl, 4-chlorobutyl, 3-bromopropyl, and a combination thereof.

12. The conjugated biomolecule of claim 11, wherein the electrophilic warhead is selected from a group consisting of electrophilic warhead may include, but is not limited to, 3-iodo-1-phenylpropan-1-one, 3-iodo-1-(p-tolyl)propan-1-one, 4-iodo-1-phenylbutan-2-one, 1-iodooctan-3-one, 1-iodopentan-3-one, 3-iodo-1-(4-methoxyphenyl)propan-1-one, 3-iodo-1-(4-(trifluoromethyl)phenyl)propan-1-one, 3-iodo-1-(3-methoxyphenyl)propan-1-one, 3-iodo-1-(3-(trifluoromethyl)phenyl)propan-1-one, 1-iodo-4-methylpentan-3-one, N-(2-hydroxyethyl)-6-iodo-N,N-dimethyl-4-oxohexan-1-aminium bromide, and a combination thereof.

13. The conjugated biomolecule of claim 11, wherein the electrophilic warhead is bound to an N-terminus of the biomolecule.

14. The conjugated biomolecule of claim 11, wherein the electrophilic warhead is bound to a C-terminus of the biomolecule.

15. The conjugated biomolecule of claim 11, wherein the biomolecule comprises an average molecular weight of at least about 1,000 g/mol.

16. The conjugated biomolecule of claim 15, wherein the biomolecule comprises an average molecular weight of at least about 20,000 g/mol.

17. The conjugated biomolecule of claim 11, wherein the biomolecule comprises a nucleic acid, a protein, a peptide, or the like.

18. A method of imaging live cells, the method comprising
incubating the live cells with a bioconjugation warhead for a sufficient time to allow for metabolic incorporation of the bioconjugation warhead;
electrochemically promoting the formation of a conjugated biomolecule within the live cells; and
imaging the live cells comprising the conjugated biomolecule and the dye.

19. The method of claim 18, wherein the dye comprises a fluorescent dye.

20. The method of claim 19, wherein the fluorescent dye comprises fluorescein thiosemicarbazide.
